(19) United States
(12) Patent Application Publication
(10) Pub. No.: US 2005/0048040 A1
(43) Pub. Date: Mar. 3, 2005

(54) Methods for improving pancreatic islet cell transplantation

(75) Inventors: Alvin C. Powers, Brentwood, TN (US); Marcela Brissova, Nashville, TN (US); Maureen A. Gannon, Nashville, TN (US)

Correspondence Address: FULBRIGHT & JAWORSKI L.L.P., 600 CONGRESS AVE., SUITE 2400, AUSTIN, TX, 78701, US

(73) Assignee: Vanderbilt University

(21) Appl. No.: 10/776,669

(22) Filed: Feb. 11, 2004

Related U.S. Application Data
(60) Provisional application No. 60/448,625, filed on Feb. 18, 2003.

(51) Int. Cl. A61K 48/00

(52) U.S. Cl. 424/93.21; 424/93.7

(57) ABSTRACT

The present invention provides for improved methods of transplanting insulin-producing cells for the treatment of diabetes. The methods involve the manipulation of endothelial cell quantity and quality in the transplated material, arising from the inventor's discovery that donor endothelial cells contribute to the revascularization of grafted material.

The present invention claims benefit of priority to U.S. Provisional Serial No. 60/448,625, filed Feb. 18, 2003, the entire contents of which are hereby incorporated by reference.

The U.S. Government owns rights in the present invention pursuant to grant number DK62641 and DK63439 from the National Institutes of Health and VA Merit Review Grant.

### BACKGROUND OF THE INVENTION

1. Field of the Invention

The present invention relates generally to the field of human cell biology and pathology, and more particularly to the areas of diabetes, transplant biology and angiogenesis. Specifically, it provides methods for improving the ability of insulin-producing cells to survive transplant into a recipient in need therefor. The methods disclosed rely in part upon the discovery that endothelial cells from the donor tissues are in part responsible for the revascularization necessary for insulin-producing cell survival.

2. Description of Related Art

Cells of neuroendocrine origin generally have the capacity to synthesize and secrete one or more polypeptide products in a regulated manner. Neuroendocrine cells by definition have sorting mechanisms, whereby a given polypeptide or protein, destined for secretion, is targeted to the regulated secretory pathway or the default constitutive secretory pathway. These cells also have processes for achieving secretory protein maturation, which generally involves protein folding, disulfide bond formation, glycosylation, endoproteolytic processing as well as other types of post-translational modifications. Neuroendocrine cells also exhibit controlled release of the secretory protein or polypeptide, most often in response to one or more external signaling molecules, or “secretagogues,” and thus have regulatory pathways allowing the cells to secrete a desired product from the secretory storage granules in response to physiological or pharmacological stimuli.

One of the best known examples of neuroendocrine cells are the β-cells of the islets of Langerhans in the pancreas, which cells secrete insulin in response to secretagogues such as amino acids, glyceraldehyde, free fatty acids, and, most prominently, glucose. The capacity of normal islet α-cells to sense a rise in blood glucose concentration and to respond to elevated levels of glucose by secreting insulin is critical to the control of blood glucose levels. Increased insulin secretion in response to a glucose load prevents hyperglycemia in normal individuals by stimulating glucose uptake into peripheral tissues, particularly muscle and adipose tissue.

Individuals in whom islet β-cell function is impaired suffer from diabetes. Insulin-dependent diabetes mellitus, or IDDM (also known as Juvenile-onset or Type I diabetes), represents approximately 10% of all human diabetes. IDDM is distinct from non-insulin dependent diabetes (NIDDM) in that only IDDM involves specific destruction of the insulin producing β-cells of the islets of Langerhans. The destruction of β-cells in IDDM appears to be a result of specific autoimmune attack, in which the patient's own immune system recognizes and destroys the β-cells, but not the surrounding α-cells (glucagon producing) or δ-cells (somatostatin producing) that comprise the islet.

Treatment for IDDM is still centered around self-injection of insulin once multiple tims per day—clearly an inconvenient and imprecise solution—and thus the development of new therapeutic strategies is highly desirable. The possibility of islet or pancreas fragment transplantation has been investigated as a means for permanent insulin replacement (Lacy, 1995; Vajkoczy et al., 1995). However, this approach, though initially attracting much interest, has been severely hampered by the difficulties associated with obtaining sufficient quantities of tissue, as well as the relatively low rate at which transplanted islets survive and succesfully graft. One of the key problems is the difficulty associated with rapid and efficient establishment of a proper vasculature in the recipient to support the transplanted islets. Pancreatic islets in the native pancreas are highly vascularized, but islet isolation prior to islet transplantation severs arterial and venous connections. These connections must be reestablished for the transplanted islets to survive.

Thus, it is clear that there remains a critical need to establish alternatives to treatment of diabetes by injectable insulin. One of these alternatives—islet transplantion—is a promising approach that has yet to become a widely utilized therapy. Therefore, methods for improving islet transplant success rates are highly sought after.

### SUMMARY OF THE INVENTION

The present invention overcomes these and other drawbacks inherent in the prior art by providing methods for the transplant of insulin-producing cells in the context of treating diabetes. The methods rely on improving the quality and/or quantity of endothelial cells in a transplantable preparation of insulin-producing cells, and in that way improve the revasculaturization and function of transplanted islets.

Thus, in accordance with the present invention, there is provided a method for transplanting insulin-producing cells comprising (a) providing a cell preparation comprising insulin-producing cells; (b) performing one or more steps that increase the quantity and/or quality of endothelial cells in the preparation; and (c) transplanting cells of the preparation into a host mammal. Increasing the quantity of endothelial cells may comprise one or more of (i) adding endothelial cells to the preparation, (ii) culturing endothelial cells of the preparation, or (iii) contacting endothelial cells of the preparation with one or more growth factors. Increasing the quality of endothelial cells may comprise one or more of (i) genetically-modifying endothelial cells in the preparation, (ii) culturing endothelial cells of said preparation, or (iii) contacting endothelial cells of the preparation with one or more growth factors. Step (c) may comprise transplanting by injection into the host hepatic portal vein or other suitable site.

The insulin-producing cells may comprise islet cells, hepatocytes, neurons, myocytes or genetically engineered cells, and may be obtained from the host, from a distinct living donor, or from a cadaver. The endothelial cells may be intra-islet endothelial cells, stem cells or bone marrow cells, and may be obtained from the host, from a distinct living donor, or from a cadaver.

The method may further comprise the step of treating the donor or host with a pro-angiogenic composition, the step of treating the host with an immunosuppressive agent, the step of providing insulin to the host, the step of further comprising monitoring insulin levels in the host, and/or the step of further comprising monitoring glucose levels in the host. The method may also further comprise performing at least step (c) a second time, performing at least steps (b) and (c) a second time, or performing steps (a)-(c) a second time.

The method may further comprise genetically modifying an endothelial cell provided in the preparation or provided from another source. The genetic modification may be facilitated by viral or non-viral transformation. The goal of the genetic modification may be to immortalize a cell or to cause it to express one or more heterologous proteins, or to overexpression one or more homologous proteins. The host may be a human.

In another embodiment, there is provided a method for enhancing the effectiveness of transplantable insulin-producing cell preparation comprising increasing the quantity and/or quality of endothelial cells in the preparation. Increasing the quantity of endothelial cells in the preparation may comprise one or more of (i) adding endothelial cells to the preparation, (ii) culturing endothelial cells of the preparation, or (iii) contacting endothelial cells of the preparation with one or more growth factors. Increasing the quality of endothelial cells may comprise one or more of (i) genetically-modifying endothelial cells in the preparation, (ii) culturing endothelial cells of the preparation, or (iii) contacting endothelial cells of the preparation with one or more growth factors. The insulin-producing cells may comprise islet cells, hepatocytes, neurons, myocytes or genetically engineered cells, and the endothelial cells may comprise intra-islet endothelial cells, stem cells or bone marrow cells. The method may further comprise genetically modifying an endothelial cell provided in the preparation or provided from another source, for example, non-viral or viral transformation with a gene that promotes cell growth, cell differentiation or cell survival.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D—Expression of endothelial cells markers in isolated islets and pancreas of Flk-1^{wt/lacZ} mice. FIG. 1A-A freshly isolated islet from a Flk-1^{wt/wt} mouse was immunostained (Flk-1 (red), somatostatin (blue)) and optically sectioned. 3-D reconstruction was applied to a series of 90 optical sections (40× magnification). FIG. 1B-X-gal staining in the whole mount Flk-1^{wt/lacZ} pancreas as viewed by light microscopy (arrowheads point to islets, 40× magnification). FIGS. 1C and D—Detection of β-galactosidase activity and endogenous Flk-1 expression in consecutive 10 μm sections of Flk-1^{wt/lacZ} pancreas (20× magnification).

FIGS. 2A-I. Detection of intra-islet endothelial cells in revascularized grafts of mouse islets. FIGS. 2A-C—Flk-1^{wt/wt} islets transplanted into Flk-1^{wt/wt} recipients. FIGS. 2D-F—Flk-1^{wt/wt} islets transplanted into Flk-1^{wt/lacZ} recipients. FIGS. 2G-I-Flk-1^{wt/lacZ} islets transplanted into NOD-SCID recipients. The kidneys bearing the islet grafts were retrieved 3-5 weeks after transplantation (insulin (Ins, green), Flk-1 (red)). Dashed line in FIGS. 2C, 2F and 2I shows a boundary between graft and kidney cortex (kidney cortex below dashed line). FIGS. 2A, 2D and 2G—40× magnification; all other panels—20× magnification.

FIGS. 3A-F. 3-D reconstruction of vasculature within the revascularized graft of Flk-1^{wt/lacZ} islets transplanted into NOD-SCID recipient. Sixty μm sections of the kidneys bearing islet grafts were optically sectioned and 3-D reconstructed. Both donor and recipient endothelial cell were labeled for PECAM-1 (green) and β-galactosidase antibody was used to visualize donor-specific endothelial cells (β-gal, red). Islet graft was identified by staining for insulin (Ins, blue). FIGS. 3A-D and FIGS. 3E & 3F (FIG. 3F is an enlargement of highlighted area in FIG. 3E)—two different fields of view in the same islet graft; 40× magnification. FIG. 3D is a merge of the images in FIGS. 3A-C. Only endothelial cell markers are shown in FIGS. 3E and 3F. Dashed line in FIGS. 3A-E shows a boundary between graft and kidney cortex (kidney cortex to the right of dashed line). White arrows point to blood vessels derived from either donor or recipient endothelial cells; magenta arrows point to chimeric blood vessels.

FIGS. 4A-F. 3-D reconstruction of vasculature within the revascularized graft of human islets transplanted into NOD-SCID recipient. Sixty μm sections of the kidneys bearing islet grafts were optically sectioned and 3-D reconstructed. Donor and recipient endothelial cells were labeled with species-specific antibodies to CD31; mouse CD31 (mPECAM-1, green), human CD31 (hCD31, red), and insulin (blue); 60× magnification. FIG. 4D is a merge of the images in FIGS. 4A-C. FIG. 4E is an enlargement of area highlighted in FIG. 4D without insulin staining. Dashed line in FIGS. 4A-D shows a boundary between graft and kidney cortex (kidney cortex above dashed line). Arrows in FIGS. 4D and 4E point to possible vascular connections of human endothelial cells with the host vasculature revealed by examination of human islet grafts in three dimensions.

FIGS. 5A-D. Intra-islet endothelial cells become functional part of revascularized graft. NOD-SCID mice bearing Flk-1^{wt/lacZ} islet grafts were infused with endothelium-binding FITC-conjugated tomato lectin (lectin, red) 5 weeks after transplantation. Subsequently, the sections were labeled with antibodies to insulin (Ins, green) and β-galactosidase (β-gal, blue). Ten-μm sections of the kidneys bearing islet grafts were optically sectioned and 3-D reconstructed. FIGS. 5A and 5B—40× magnification, FIGS. 5C and 5D—60× magnification. Endothelial cells double positive for lectin and β-galactosidase (purple) in FIGS. 5B and 5D indicate blood flow through donor-derived capillaries.

FIG. 6—New Mechanism for Revascularization of Transplanted Islets.

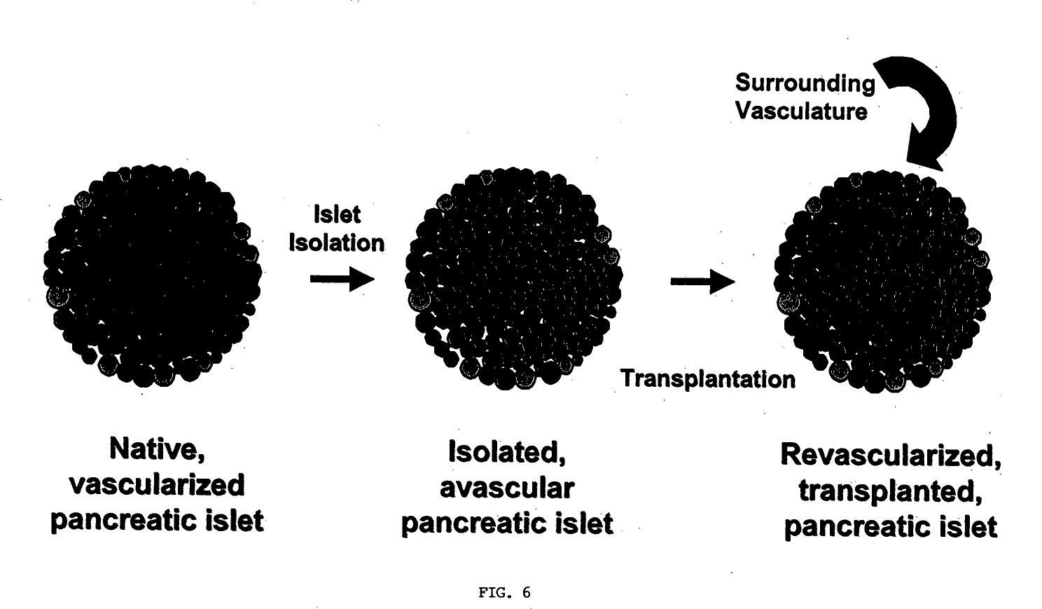

FIGS. 7A-B—Transplanted islets have reduced vascularity. The graph on the left shows that islets transplanted into the spleen, liver, or kidney have a lower vessel density than islets in the native pancreas. The graph on the right shows that freshly isolated islets have a vessel density more similar to islets within the pancreas than pancreatic exocrine tissue. While the number of vessels in freshly isolated islets is similar to islets in the pancreas, other vascular parameters of these islets are abnormal (see FIG. 8) and are more similar to those parameters in pancreatic exocrine tissue.

FIGS. 8A-C—Transplanted islets have reduced vascularization parameters. Transplanted islets have less vessel area, vessel branching, and vessel perimeter compared to islets within the pancreas; these parameters more similar to exocrine tissue.

FIG. 9—Endothelial cell number declines in culture. The graph shows beta-galactosidase (y-axis) as a function of the length of culture α-axis) in two different types of media (orange and blue bars). Note that compared to freshly isolated islets (pink bar), beta-galactosidase, a marker of intra-islet endothelial cells, declines in culture and that the EBM media preserves endothelial cell health more effectively.

FIG. 10—Endothelial cells become unhealthy and change morphology in culture. Mouse islets were cultured in for the number of hours noted below each figure. At the time, the islets were examined by confocal microscopy with staining for insulin and an endothelial cell marker (red). Note the change in endothelial pattern during culture.

### DESCRIPTION OF ILLUSTRATIVE EMBODIMENTS

In the development of alternative therapeutic strategies for treating diabetes (improved over insulin injection), the transplant of insulin-producing cells continues to attract considerable attention. However, there have been only limited successes in such transplants. A major reason for the lack of success is the length of time required for the transplanted islets to develop a supporting vasculature. Current dogma states that new vessel formation following islet transplantation arises from recipient endothelial cells, cells that line all blood vessels, as well as existing recipient blood vessels. Vajkoczy et al. (1995). It is thought that endothelial cells and blood vessels surrounding the islet transplant grow into the transplanted islets by a process known as angiogenesis.

Since isolated islets retain intra-islet endothelial cells, the present inventors examined the ability of these endothelial cells to contribute to revascularization of transplanted islets. Using a mouse model of islet transplantation, they surprisingly found that islet revascularization arises from endothelial cells within the transplanted islets, as well as from endothelial cells from the transplant recipient. This observation indicates that improving the quality and/or quantity of donor (as opposed to host) endothelial cells will improve revascularization and the survival of transplanted islets.

I. Endothelial Cells

Endothelial cells for use in the present invention may be derived from any part of the vascular tree. They may be undifferentiated (stem cells) or differentiated (bone marrrow cells). They also may come from large and small veins and arteries, from capillaries, from specialized vascular areas such as the umbilical vein of newborns, or from vascularized solid tumors. A particular cell of interest will be the intra-islet endothelial cell, given the convenience of being present in a transplantable islet cell preparation, and the likelihood of compatibility with islet vasculaturization.

Another convenient source of endothelial cells will be human umbilical veins. Hence, a number of media have been developed using cells from this source. However, it is quite likely that with little or no modification, these media will be useful for culturing endothelial cells from most, if not all, of the above sources. Such media (HUVEC culture medium) are commercially available from Sigma (St. Louis, Mo.).

II. Insulin-Producing Cells

Secretory cells, especially neuroendocrine cells, have several endogenous functions that make them uniquely suited for production of a wide range of proteins, including secreted peptide hormones. These specialized functions are encompassed by the regulated secretory pathway. The regulated secretory pathway embodies the secretory granules of neuroendocrine cells which serve as the site of maturation and storage of a large class of peptide hormones with profound biological functions. Proper biological function of the peptides is due both to their secretion in a regulated and titratable manner as well as a complex set of post-translational modifications resulting in the final biologically active product. Moreover, complete processing requires sufficient levels of the processing enzymes as well as sufficient retention of the maturing peptides. In this way, physiological signals leading to the release of the contents of the secretory granules ensures release of fully processed, active proteins.

Examples of insulin-producing cells for use in conjunction with the present invention are discussed below in detail, and shown in Table 1 (Pearse and Takor, 1979; Nylen and Becker, 1995).

A. Pancreatic and Pituitary Cells

The origin of the insulin-producing cells for use in the present invention thus include a variety of cells that provide both the necessary capacity to producing insulin in response to physiologic signals, and the machinery necessary to process and release insulin through the proper secretory pathway. Clearly, pancreatic β-cells and pituitary cells are preferred for use in the present invention, with β-cells being more preferred. In other aspects of the present invention, cells from primary human tissues are preferred for use as the starting human neuroendocrine cells. Human organs can be obtained from autopsies through nonprofit organ procurement centers. High quality human islets are available, for example, from Dr. Camillo Ricordi of the University of Miami Medical Center, an islet transplant surgeon who supplies human islets to scientists throughout the United States. Automated methods for isolation of human pancreatic islets have been established (Ricordi et al., 1988; incorporated herein by reference).

B. Fetal Cells

In certain aspects of the present invention, fetal cells are preferred for use as the starting insulin-producing cells. Human fetal organs, such as fetal pancreases at 18 to 24 gestational weeks, can be obtained through nonprofit organ procurement centers, with patient consent for tissue donation being obtained. Dissection of specific organs from the fetuses is often done at the procurement centers. Isolation of fetal pancreases and islets is performed by established techniques (Otonkoski et al., 1993; incorporated herein by reference).

C. Cells from Resected Neuroendocrine Tumors

Explanted tumor samples from surgically resected tumors are another starting material. More preferred are insulinomas and pituitary tumors. Two exemplary insulinomas have been reported (Gueli et al, 1987; Cavallo et al., 1992). In general, these cells are likely to have difficulty processing proinsulin to insulin (Comi and Gorden, 1995), and often express significant levels of other select hormones, such as glucagon and somatostatin.

D. Hepatocytes and Myocardial Cells

Other suitable insulin-producing cells include genetically modified hepatocytes and myocytes that are rendered capable of producing insulin. The may be obtainined from the transplant recipient, a living donor or a recently deceased cadaver.

TABLE 1
Neuroendocrine Cell Types

| Neuroendocrine cell | Endogenous hormone | Tissue specific promoter | Context specific promoter | Associated Tumors |
|---|---|---|---|---|
| Hypothalamic/pituitary cells | | | | |
| Corticotropes | ACTH, LPH | | POMC (V01510, K02406) | Corticotrope adenoma |
| Somatotropes | Growth Hormone | Growth Hormone (J03071, | | GH producing adenoma |
| | | K00470) | | |
| Melanotropes | alpha-MSH, endorphins | | POMC (V01510, K02406) | |
| Lactotropes | Prolactin | Prolactin (X00368, L33865) | | Prolactin adenomas |
| Thyrotropes | Thyroid-stimulating | TSH (M23669, S70587) | α-glycoprotein (L05632) | Thyrotrope adenomas |
| | hormone | | | |
| Gonadotropes | Follicle Stimulating hormone | FSH (M16646), LH (X00264) | α-glycoprotein (L05632) | Gonadotrope adenoma |
| | Leuteinizing hormone | | α-glycoprotein (L05632) | |
| Adrenal medulla | enkephalins, dynorphin | NPY (M14296) | | |
| | NPY, bombesin | | | |
| Thyroid C Cell | Calcitonin, CGRP, | | Calcitonin (X15943), | Thyroid carcinomas |
| | Somatostatin, Bombesin | | Somatostatin (J00306) | |
| Parathyroid Chief Cells | Parathyroid hormone | Parathyroid hormone (J00301) | | parathyroid adenomas |
| Pulmonary neuroendocrine | Calcitonin, Bombesin, | | Calcitonin (X15943) | Small Cell Lung |
| cells/K cells | CGRP, cholecystokinin, | | | Carcinoma |
| | endothelin | | | |
| Gastric G cells | Gastrin, enkephalin | | Gastrin (X00183) | Gastrinoma |
| Gastric D cells | Somatostatin | | Somatostatin (J00306) | Somatostatinoma |
| Enteroneuroendocrine cell | | | | |
| types | | | | |
| L cells | Glucagon Family peptides | Peptide YY (L25648, D13897) | Glucagon (X03991) | Glucagonoma |
| | Peptide YY | | | |
| D cells | Somatostatin | | Somatostatin (J00306) | Somatostatinoma |
| S cells | Secretin | Secretin | | |
| G cells | Gastrin | | Gastrin (X00183) | Gastrinoma |
| K cells | GIP | GIP (M31674) | | |
| I cells | CCK | CCK (L29399, M15843, | | Enteroneurocrinomas |
| | | N00050) | | |
| N cells | Neurotensin | Neurotensin (S47339) | | Neurotensinoma |
| H cells | VIP | | VIP (M33027, M37460) | VIPomas |
| Enterochromafin cells | Motilin, Substance P, | Motilin (X15392, Y07505, | Substance P (M68906) | Carcinoid tumors |
| | Substance K | M30277) | | |
| Pancreatic Cells | | | | |
| β-cells | Insulin, amylin | Insulin (V00565), amylin | | Insulinoma |
| | | (L08226) | | |
| α-cells | Glucagon, Corticotropin | Glucagon (X03991), | Glucagon (X03991) | Glucagonoma |
| | releasing hormone | Corticotropin releasing | | |
| | | hormone (X55962) | | |
| PP cells | Pancreatic polypeptide | Pancreatic polypeptide | | PPoma |
| | | (M11726) | | |
| δ-cells | Somatostatin | | Somatostatin (J00306) | Somatostatinoma |
| G cells | Gastrin | | Gastrin (X00183) | Gastrinoma |
| Carotid I cell | Substance P, enkephalin | | Substance P (M68906) | Carcinoid tumors |
| Urigenital tract Merkel cells | Calcitonin, bombesin, VIP | VIP (M33027, M37460) | VIP (M33027, M37460) | VIPomas |
| Syncytiotrophoblast cell | chorionic-gonadotropin | chorionic-gonadotropin | | Trophoblast tumors |
| | | (M13504) | | |
| Neurons | | | | |
| Supraoptic and | Vasopressin, oxytocin | Vasopressin (X62890), | | |
| paraventricular nuclei | | oxytocin (M11186) | | |
| Sympathetic ganglion | VIP, enkephalin | | VIP (M33027, M37460) | VIPomas |
| Paraganglia | enkephalin | | | |

III. Cell Culturing

A. Culture Conditions

Primary cells (insulin-producing and endothelial cells) may be expanded by established culture conditions. For example, β-cells can be cultured and even induced to divide as described (Clark and Chick, 1990; Beattie et al. 1991; Hayek et al. 1995; each incorporated herein by reference). Endothelial cells may be cultured according to methods disclosed by Nitsri et al. (2002).

More generally, culture conditions may involve manipulating the following cell culture variables: media growth/survival factors (such as IGF-1, growth hormone, prolactin, PDGF, hepatocyte growth factor, and transferrin), media differentiation factors (such as TGF-β), media lipids, media metabolites (such as glucose, pyruvate, galactose, and amino acids), media serum (percentage serum, serum fraction, species of serum), gaseous exchange (ratio atmospheric O₂:CO₂, and media volume), physical form of the islets prior to plating (whole, dispersed, or cell sorted islet cells), and extracellular substrate for cellular attachment (such as laminin, collagen, matrigel, and HTB-9 bladder carcinoma derived matrix).

B. Defined Media

Media comprising one or more growth factors that stimulate the growth of the target cell and do not substantially stimulate growth of distinct cells in the cell population; i.e., act to induce preferential growth of the target cells rather than faster-growing, more hardy cells in the population, may be used to deplete fibroblasts. Examples include defined serum free conditions used for β-cells (Clark et al., 1990; incorporated herein by reference), or inclusion of growth or differentiation factors known to allow preferential growth of β-cells (WO95/29989; PCT/US99/00553; incorporated herein by reference). A commercially available medium, EGM™ Endothelial Cell Medium (Cambrex) is a preferred defined media.

C. Proliferation

Cells also may be induced to proliferate by initial infection with adenovirus or adeno-associated virus (AAV) comprising a gene that induces cellular proliferation, the gene being under the control of a promoter specific for the regulated secretory cell. The cells may alternatively be induced to proliferate by growth on a stimulatory cell matrix.

V. Growth Factors

In accordance with the present invention, one may choose to treat isolated or transplanted cells (e.g., though genetic engineering) with growth factors that will stabilize or expand the transplanted cell population. Such growth factors include, but are not limited to, vascular endothelial growth factor (VEGF), angiopoietin-1 or -2, fibroblast growth factor (various isoforms), endothelial cell growth factor and endothelial cell attachment factor.

VI. Genetic Engineering of Endothelial and Insulin-Producing Cells

In one aspect of the invention, it may prove useful to modify endothelial and/or insulin-producing cells of the present invention to better adapt them for use in accordance with the methods described herein.

A. Engineered Endothelial Cells

Endothelial cells are specialized cells which form the lining of the heart and the blood vessels. Because of their direct contact with the circulating blood, a number of proposals have been made to genetically engineer these cells and use them as “in vivo” drug delivery systems. See, for example, Culliton (1989) and Zwiebel et al. (1989) (transfer of a human adenosine deaminase gene and a rat growth hormone gene to aortic endothelial cells using a retroviral vector and demonstration of the secretion of rat growth hormone from such cells after seeding onto a synthetic vascular graft).

Genetic engineering of endothelial cells has been performed by a number of workers in the art. For example, Nabel et al. (1989) describe experiments in which a gene for the marker protein β-galactosidase was transferred to endothelial cells using a retroviral vector and the thus modified cells were seeded onto the walls of an artery in vivo using a double balloon catheter to isolate the section of the artery where the seeding took place. They reported that up to four weeks after surgery, the seeded arteries were found to contain endothelial cells which expressed β-galactosidase.

Wilson et al. (1989) describe similar work wherein a β-galactosidase gene was transferred to endothelial cells using a retrovirus, the modified cells were seeded onto synthetic grafts, and the grafts were implanted in the carotid arteries of dogs. Five weeks later, the grafts were removed and found to still contain the genetically modified endothelial cells along their luminal surfaces.

Along these same lines, Dichek et al. (1989) describe the seeding of stainless steel stents with genetically engineered endothelial cells carrying in some cases a β-galactosidase gene and in others a human tissue-type plasminogen activator (TPA) gene. See also PCT WO 90/06997 (transfer of β-galactosidase, rat growth hormone, and human adenosine deaminase, CD-4, and TPA genes to endothelial cells and seeding of silicon coated polyurethane grafts and stainless steel stents with genetically engineered cells); and Zweibel et al. (1989).

Direct in vivo transformation of arterial endothelial cells using retroviral particles or plasmid carrying liposomes is described in Nabel et al. (1990). β-galactosidase was again used as a marker protein, and evidence of transformation could be found 21 weeks after transfection.

Attention is also directed to U.S. Pat. Nos. 5,336,615, 5,587,301, and 5,783,669, each of which is incorporated by reference.

B. Engineered Insulin-Producing Cells

In certain embodiments, it may be useful to genetically engineer insulin-producing cells prior to use with the present invention. U.S. Pat. No. 5,427,940 desribes recombinant cells that secrete insulin in response to glucose. The generation of such artificial β-cells is achieved through the introduction of one or more genes selected from the insulin gene, the glucokinase gene and the GLUT-2 glucose transporter gene, so as to provide an engineered cell having all three of these genes in a biologically functional and glucose-responsive configuration. While the insulin-producing cells of the present invention would not necessarily need to be engineered to achieve insulin production, this sort of manipulation is instructive on the manner in which cells of this kind can be engineered.

The possibility of producing insulin-producing cell lines by cell fusion techniques are described in U.S. Pat. No. 4,195,125 and PCT application WO 87/05929. PCT application WO 87/05929 demonstrates generation of human liver cell lines. A number of insulinoma cell lines have been established by expression of SV40 large T antigen in β-cells of transgenic animals under control of the rat insulin promoter/enhancer. Early lines produced by this method, such as βTC-1 and βTC-3, were shown to be glucose responsive, but with maximal secretion occurring at subphysiological glucose levels (Efrat et al., 1988). Two lines designated MIN6 and MIN7, which have different phenotypes and different insulin secretory profiles despite being generated with the identical insulin promoter-SV40 large T antigen construct, have been described (Miyazaki et al., 1990). A separate rodent cell line derived by the transgenic T-antigen approach termed PTC-6, which, like MIN6, performs glucose stimulated insulin secretion with a concentration dependence similar to islets, and retains GLUT-2 and glucokinase expression in lieu of hexokinase and GLUT-1, has been reported (Efrat et al., 1993). Recently, the same group has used soft agar cloning of βTC-6 cells to establish a cell line (βTC6-F7) which retains glucose responsiveness at physiological glucose concentrations through at least 55 passages (Knaack et al., 1994). Isolated reports have also suggested that human islet cell lines could perhaps be generated by providing the cell with an oncogene under the control of a heterologous, or tissue-specific promoter (PCT application, WO 91/09939; Soldevila et al. 1991).

C. Recombinant Engineering Technologies

Recombinant vectors form important further aspects of the present invention. The term “expression vector or construct” means any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of a RNA into a gene product. In other embodiments, expression only includes transcription of the nucleic acid, for example, to generate antisense or ribozyme constructs.

Particularly useful vectors are contemplated to be those vectors in which the coding portion of the DNA segment, whether encoding a full length protein or smaller peptide, is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned”, “under control” or “under transcriptional control” means that the promoter is in the correct location and orientation in relation to the nucleic acid to control RNA polymerase initiation and expression of the gene.

The promoter may be in the form of the promoter that is naturally associated with a particular gene, as may be obtained by isolating the 5′ non-coding sequences located upstream of the coding segment or exon, for example, using recombinant cloning and/or polymerase chain reaction (PC™) technology, in connection with the compositions disclosed herein (PCR technology is disclosed in U.S. Pat. No. 4,683,202 and U.S. Pat. No. 4,682,195, each incorporated herein by reference).

In other embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a particular gene in its natural environment. Such promoters may include promoters normally associated with other genes, and/or promoters isolated from any other bacterial, viral, eukaryotic, or mammalian cells.

Naturally, it will be important to employ a promoter that effectively directs the expression of the DNA segment in the cell type chosen for expression. The use of promoter and cell type combinations for protein expression is generally known to those of skill in the art of molecular biology, for example, see Sambrook et al. (1989), incorporated herein by reference. The promoters employed may be constitutive, or inducible, and can be used under the appropriate conditions to direct high level expression of the introduced DNA segment, such as is advantageous in the large-scale production of recombinant proteins or peptides.

At least one module in a promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

1. Secretory Cell-Specific Promoters

The promoter is required to express the transforming genetic construct to a degree sufficient to effect transformation of a target cell type amongst a population of different cell types such that the transformed target cell results in the generation of a stable human regulated secretory cell.

Promoters can be classified into two groups, ubiquitous and tissue- or cell-specific. Ubiquitous promoters activate transcription in all or most tissues and cell types. Examples of ubiquitous promoters are cellular promoters like the histone promoters, promoters for many metabolic enzyme genes such as hexokinase I and glyceraldehyde-3-phosphate dehydrogenase, and many viral promoters such as the cytomegalovirus promoter (CMVp) and the Rous sarcoma virus promoter (RSVp). In certain aspects of the present invention, these promoters are appropriate for use with the immortalizing constructs described herein, as well as finding use in additional aspects of the present invention.

Tissue- or cell-specific promoters activate transcription in a restricted set of tissues or cell types or, in some cases, only in a single cell type of a particular tissue. Examples of stringent cell-specific promoters are the insulin gene promoters which are expressed in only a single cell type (pancreatic β-cells) while remaining silent in all other cell types, and the immunoglobulin gene promoters which are expressed only in cell types of the immune system. Endothelial cell promoters of interest in the present invention include PECAM, flk-1, flt-1 and Tie-2.

The promoter may also be “context specific” in that it will be expressed only in the desired cell type and not in other cell types that are likely to be present in the population of target cells, e.g., it will be expressed in β-cells, but not in α- or δ-cells, when introduced into intact human islets. For example, an insulin promoter targets the expression of a linked transforming oncogene selectively to β-cells of a human islet preparation even though many other contaminating cell types exist in the preparation.

a. β-Cell-Specific Promoters

It has been documented that the two rat insulin gene promoters, RIP1 (GenBank accession number J00747) and RIP2 (GenBank accession number J00748), as well as the human insulin promoter (HIP; GenBank accession number V00565), direct stringent cell-specific expression of the insulin gene in rodent β-cell insulinoma lines (German et al., 1990), primary islet cells (Melloul et al., 1993), and in β-cells of transgenic mice (Efrat et al., 1988).

As the sequence and position of the functional promoter elements are well conserved between HIP, RIP1 and RIP2, the transcription factors that interact with these elements are likely to be conserved across species. In fact, HIP can direct cell-specific expression of linked genes in rodent β-cell lines and rat primary islets, albeit, at a somewhat lower level than that observed for RIP1 (Melloul et al., 1993). Melloul et al. (1993) demonstrated that the isolated 50-bp RIP1 FAR/FLAT minienhancer (FF), an essential promoter element for RIP1 activity, could express a linked reporter gene in both adult rat and human islet cells. Furthermore, FF activity could be substantially induced by increased concentrations of glucose in both species of adult islets. Additional results from gel-shift studies strongly suggested that the same or similar β-cell-specific transcription factor(s) from both adult rat and human islet cell nuclear extracts bound to conserved sequences contained within both the RIP1 FF and the analogous region of HIP.

b. Further Neuroendocrine Cell-Specific Promoters

As used herein in the context of the present invention, the term “neuroendocrine cell specific promoter” is used to define a promoter that is specific for one or more types of neuroendocrine cells. Thus, in certain aspects of the present invention, a promoter that specifically expresses of at least a first construct in one, two, three, four, a plurality or all neuroendocrine cell types, but not in other, non-neuroendocrine cell types, is contemplated for use.

Exemplary of this class of promoter are the glucagon promoter, GenBank accession number X03991; growth hormone promoter, GenBank accession numbers J03071 and K00470; POMC gene promoter, GenBank accession numbers V01510 and K02406; calcitonin promoter, GenBank accession number X15943; the GIP gene promoter, GenBank accession number M31674, and the α-glycoprotein promoter (Genbank accession number LO5632).

2. Modified Promoters

Promoters can be modified in a number of ways to increase their transcriptional activity. Multiple copies of a given promoter can be linked in tandem, mutations which increase activity may be introduced, single or multiple copies of individual promoter elements may be attached, parts of unrelated promoters may be fused together, or some combination of all of the above can be employed to generate highly active promoters. All such methods are contemplated for use in connection with the present invention.

German et al. (1992a) mutated three nucleotides in the transcriptionally important FLAT E box of the rat insulin I gene promoter (RIP), resulting in a three- to four-fold increase in transcriptional activity of the mutated RIP compared to that of a nonmutated RIP as assayed in transiently transfected HIT cells. Also, the introduction of multiple copies of a promoter element from the E. coli tetracycline resistance operon promoter were introduced into the CMV promoter, significantly increasing the activity of this already very potent promoter (Liang et al., 1996). Additionally, part of the CMV promoter, which has high but short-lived transcriptional activity in dog myoblasts, was linked to the muscle-specific creatine kinase promoter (MCKp), which has weak but sustained expression in dog myoblasts, resulting in a hybrid promoter that sustained high-level expression for extended periods in dog myoblasts.

3. Multimerized Promoters

Several modified rat insulin promoters (modRIP) containing multimerized enhancer elements have been engineered. The currently preferred modRIP contains six multimerized repeats of a 50 base pair region of the cis acting enhancer of RIP, placed upstream of an intact copy of RIP. These novel promoters have been shown to direct expression of transgenes in stably engineered β-cell lines at levels above those attained with unmodified insulin promoters and, in some cases, approaching the levels achieved with the cytomegalovirus promoter (CMVp). CMVp is one of the strongest activating promoters known, but in a very non-tissue specific manner.

4. Additional Promoters and Other Expression Elements

The particular promoter that is employed to control the expression of a nucleic acid is not believed to be critical, so long as it is capable of expressing the nucleic acid in the targeted cell. Thus, as human cells are being targeted, it is preferable to position the nucleic acid coding region adjacent to and under the control of a promoter that is capable of being expressed in a human cell. Generally speaking, such a promoter might include either a human or viral promoter. Preferred promoters include those derived from HSV, including the HNF1α promoter. Another preferred embodiment are the tetracycline controlled promoters.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters which are well-known in the art to achieve expression of a transgene is contemplated as well, provided that the levels of expression are sufficient for a given purpose.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression of a transgene. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Turning to expression, once a suitable clone or clones have been obtained, whether they be cDNA based or genomic, one may proceed to prepare an expression system. The engineering of DNA segment(s) for expression in human neuroendocrine cells may be performed by techniques generally known to those of skill in recombinant expression. It is believed that a number of different expression systems may be employed in the expression of proteins and peptides in the present invention.

Both cDNA and genomic sequences are suitable for expression in human neuroendocrine cells, as these cells will generally process the genomic transcripts to yield functional mRNA for translation into protein. Generally speaking, it may be more convenient to employ as the recombinant gene a cDNA version of the gene. It is believed that the use of a cDNA version will provide advantages in that the size of the gene will generally be much smaller and more readily employed to transfect the targeted cell than will the genomic version of the gene, which will typically be up to an order of magnitude larger than the cDNA version of the gene. However, the inventors do not exclude the possibility of employing a genomic version of a particular gene where desired.

In expression, one will also typically desire to incorporate into the transcriptional unit an appropriate polyadenylation site (e.g., 5′-AATAAA-3′) if one was not contained within the original cloned segment. Typically, the poly A addition site is placed about 30 to 2000 nucleotides “downstream” of the termination site of the protein at a position prior to transcription termination. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

It is proposed that a particular protein may be co-expressed with one or more additional selected protein(s) or polypeptide(s), wherein the proteins may be co-expressed in the same cell or one or more particular gene(s) may be provided to a human neuroendocrine cell that already has another selected protein. Co-expression may be achieved by co-transfecting the cell with two or more distinct recombinant vectors, each bearing a copy of the respective DNA. Alternatively, a single recombinant vector may be constructed to include the coding regions for the two or more proteins, which could then be expressed in cells transfected with the single vector. In either event, the term “co-expression” herein refers to the expression of two or more selected proteins in the same recombinant cell.

In certain embodiments of the invention, the use of internal ribosome binding site (IRES) elements are used to create multigene, or polycistronic, messages. IRES elements are able to bypass the ribosome scanning model of 5′ methylated Cap dependent translation and begin translation at internal sites (Pelletier and Sonenberg, 1988; Jang et al., 1988). IRES elements from two members of the picomavirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Many standard techniques are available to construct expression vectors containing the appropriate nucleic acids and transcriptional/translational control sequences in order to achieve protein or peptide expression in human neuroendocrine cells. Suitable expression vectors include, but are not limited to, bacteriophage, plasmid, cosmid, viral, or artificial chromosome (including, but not limited to, bacterial artificial chromosome (BAC), yeast artificial chromosome (YAC) and human artificial chromosome (HAC)) expression vectors.

Expression vectors for use in mammalian such cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

A number of viral based expression systems may be utilized, for example, commonly used promoters are derived from polyoma, Adenovirus 2, and most frequently Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 bp sequence extending from the HindIII site toward the BglI site located in the viral origin of replication.

It is contemplated that a protein may be “overexpressed”, i.e., expressed in increased levels relative to its natural expression in cells. Such overexpression may be assessed by a variety of methods, including radiolabeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein or peptide in comparison to the level in natural cells is indicative of overexpression, as is a relative abundance of the specific protein in relation to the other proteins produced by the host cell and, e.g., visible on a gel.

5. Oncogenes and Mutant Tumor Suppressors

In one aspect of the invention, it may be desireable to immortalize a transplated cell. Any of a large number of oncogenes (Rb, ras, myc, etc.) or anti-apoptosis genes (Bc1-X-_{L}) may be used in the context of the present invention. Where two or more immortalizing genes are provided to a cell, it may be preferable to provide genes from different functional categories, such as those that perturb signal transduction, affect cell cycle, alter nuclear transcription, alter telomere structure or function, inhibit apoptosis, or that exert pleiotropic activities.

6. Growth Factor Receptor Genes and Growth Factors

In still further preferred embodiments, the invention contemplates the use of one or more growth factor receptor genes and/or one or more growth factor genes (see above). As an example of this embodiment, human may be transformed with a construct that provides overexpression of growth hormone receptor (GenBank Accession Nos. J04811 and X06562) controlled by the modRIP (or modHIP) promoter. Cells cultured in a defined medium would then be stimulated to proliferate by the addition of growth hormone to the medium. The replicating population of cells may be further transformed by retroviral constructs that will result in stable expression of growth hormone receptor or an alternate immortalizing gene. The use of other growth promoting genes such as IGF-1 receptor (and its ligand in the medium), chicken growth hormone (cGH) and chicken growth hormone receptor (cGHr), where the cGH is specific for the cGHr, the signaling substrate of growth factor receptors (such as IRS-2 in the case of IGF-1 receptor), insulin, hepatocyte growth factor (HGF), platelet derived growth factor (PDGF), nerve growth factor (NGF), growth hormone or epidermal growth factor (EGF), could be used.

7. Other Proliferative Factors

Insulin promoter factor 1 (IPF-1; also referred to as STF-1, IDX-1, PDX-1 and βTF-1) is a homeodomain-containing transcription factor proposed to play an important role in both pancreatic development and insulin gene expression in mature β-cells (Ohlsson et al., 1993, Leonard et al., 1993, Miller et al., 1994, Kruse et al., 1993). In embryos, IPF-1 is expressed prior to islet cell hormone gene expression and is restricted to positions within the primitive foregut where the pancreas will later form. Indeed, mice in which the IPF-1 gene is disrupted by targeted knockout do not form a pancreas (Jonsson et al., 1994). Later in pancreatic development, as the different cell types of the pancreas start to emerge, IPF-1 expression becomes restricted predominantly to β-cells. IPF-1 binds to TAAT consensus motifs contained within the FLAT E and β1 elements of the insulin enhancer/promoter, whereupon, it interacts with other transcription factors to activate insulin gene transcription (Peers et al., 1994).

Additional cell-specific transcription factors contemplated for use in the present invention include, but are not limited to, CDX-3 and LMX-1 (LMX1.1 and LMX1.2) (German et al., 1992b; German et al., 1994), NKX6.1 (Inoue et al., 1997), NKX2.2 (Sussel et al., 1998), Beta-2 (Sander and German, 1997), E47/Pan-1 (Johnson et al., 1997), ISL1 (Ahlgren et al., 1997) and PAX6 (Sander et al., 1997).

8. DNA Delivery

In certain embodiments of the invention, the nucleic acid encoding the one or more product(s) of interest may be integreated into the host cell's genome. In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. All delivery methods are contemplated for use in the context of the present invention, although certain methods are preferred, as outlined below.

a. Transfection

In order to effect expression, the construct must be delivered into a cell. As described below, the preferred mechanism for delivery is via viral infection, where the construct is encapsidated in an infectious viral particle. However, several non-viral methods for the transfer of one or more immortalizing or other expression constructs into cultured mammalian cells also are contemplated by the present invention. In one embodiment of the present invention, the expression construct may consist only of naked recombinant DNA or plasmids. Transfer of the construct may be performed by any of the methods mentioned which physically or chemically permeabilize the cell membrane.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an expression construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1.

Melloul et al. (1993) demonstrated transfection of both rat and human islet cells using liposomes made from the cationic lipid DOTAP, and Gainer et al. (1996) transfected mouse islets using Lipofectamine-DNA complexes.

In certain embodiments of the present invention, the expression construct is introduced into the cell via electroporation. Electroporation involves the exposure of a suspension of cells and DNA to a high-voltage electric discharge.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner. Examples of electroporation of islets include Soldevila et al. (1991) and PCT application WO 91/09939.

In other embodiments of the present invention, the expression construct is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and erythroleukemia cells (Gopal, 1985).

Another embodiment of the invention for transferring one or more naked DNA immortalizing or other expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Gainer et al. (1996) have transfected mouse islets with a luciferase gene/human immediate early promoter reporter construct, using biolistic particles accelerated by helium pressure.

Further embodiments of the present invention include the introduction of the expression construct by direct microinjection or sonication loading. Direct microinjection has been used to introduce nucleic acid constructs into Xenopus oocytes (Harland and Weintraub, 1985), and LTK fibroblasts have been transfected with the thymidine kinase gene by sonication loading (Fechheimer et al., 1987).

In certain embodiments of the present invention, the expression construct is introduced into the cell using adenovirus assisted transfection. Increased transfection efficiencies have been reported in cell systems using adenovirus coupled systems (Kelleher and Vos, 1994; Cotten et al., 1992; Curiel, 1994), and the inventors contemplate using the same technique to increase transfection efficiencies into human islets.

Still further constructs that may be employed to deliver the one or more immortalizing or other expression construct to the target cells are receptor-mediated delivery vehicles. These take advantage of the selective uptake of macromolecules by receptor-mediated endocytosis that will be occurring in the target cells. In view of the cell type-specific distribution of various receptors, this delivery method adds another degree of specificity to the present invention. Specific delivery in the context of another mammalian cell type is described by Wu and Wu (1993; incorporated herein by reference).

Certain receptor-mediated gene targeting vehicles comprise a cell receptor-specific ligand and a DNA-binding agent. Others comprise a cell receptor-specific ligand to which the DNA construct to be delivered has been operatively attached. Several ligands have been used for receptor-mediated gene transfer (Wu and Wu, 1987, 1988; Wagner et al., 1990; Ferkol et al., 1993; Perales et al., 1994; Myers, EPO 0273085), which establishes the operability of the technique. In the context of the present invention, the ligand will be chosen to correspond to a receptor specifically expressed on the neuroendocrine target cell population.

In other embodiments, the DNA delivery vehicle component of a cell-specific gene targeting vehicle may comprise a specific binding ligand in combination with a liposome. The nucleic acids to be delivered are housed within the liposome and the specific binding ligand is functionally incorporated into the liposome membrane. The liposome will thus specifically bind to the receptors of the target cell and deliver the contents to the cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the DNA delivery vehicle component of the targeted delivery vehicles may be a liposome itself, which will preferably comprise one or more lipids or glycoproteins that direct cell-specific binding. For example, Nicolau et al. (1987) employed lactosyl-ceramide, a galactose-terminal asialganglioside, incorporated into liposomes and observed an increase in the uptake of the insulin gene by hepatocytes. It is contemplated that the one or more immortalizing or other expression constructs of the present invention can be specifically delivered into the target cells in a similar manner.

b. Viral Infection

One of the preferred methods for delivery of expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue-specific transforming construct that has been cloned therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the E3, or both the E1 and E3 regions (Graham and Prevec, 1991). In nature, adenovirus can package approximately 105% of the wild-type genome (Ghosh-Choudhury et al., 1987), providing capacity for about 2 kb of extra DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of media. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of media, is added to the carrier (50 ml) in a 250 ml Erlenmeyer flask and left stationary, with occasional agitation, for 1 to 4 h. The media is then replaced with 50 ml of fresh media and shaking initiated. For virus production, cells are allowed to grow to about 80% confluence, after which time the media is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector preferred for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Recombinant adenovirus and adeno-associated virus (see below) can both infect and transduce non-dividing human primary cells. Gene transfer efficiencies of approximately 70% for isolated rat islets have been demonstrated by the inventors (Becker et al., 1994a; Becker et al., 1994b; Becker et al., 1996) as well as by other investigators (Gainer et al., 1996).

Adeno-associated virus (AAV) is an attractive vector system for use in the human cell transformation of the present invention as it has a high frequency of integration and it can infect nondividing cells, thus making it useful for delivery of genes into mammalian cells in tissue culture (Muzyczka, 1992). AAV has a broad host range for infectivity (Tratschin, et al., 1984; Laughlin, et al., 1986; Lebkowski, et al., 1988; McLaughlin, et al., 1988), which means it is applicable for use with human neuroendocrine cells, however, the tissue-specific promoter aspect of the present invention will ensure specific expression of the transforming construct in aspects of the invention where this is desired or required. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt, et al., 1994; Lebkowski, et al., 1988; Samulski, et al., 1989; Shelling and Smith, 1994; Yoder, et al., 1994; Zhou, et al., 1994; Hermonat and Muzyczka, 1984; Tratschin, et al., 1985; McLaughlin, et al., 1988) and genes involved in human diseases (Flotte, et al., 1992; Luo, et al., 1994; Ohi, et al., 1990; Walsh, et al., 1994; Wei, et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski, et al., 1989; McLaughlin, et al., 1988; Kotin, et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975). Additional retroviral vectors contemplated for use in the present invention have been described (Osborne et al., 1990; Flowers et al., 1990; Stockschlaeder et al., 1991; Kiem et al., 1994; Bauer et al., 1995, Miller and Rosman, 1989; Miller, 1992; Miller et al., 1993; each incorporated herein by reference).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990). A preferred cell line is the PA317 cell line (Osborne et al., 1990).

A major determinant of virus titer is the number of packagable RNA transcripts per producer cell, which is dependent on the integrated proviral DNA copy number. Packaging cell lines are coated with viral envelope glycoproteins and are thus resistant to infection by virus of the same host range, but not virus of a different host range. This process is called interference. Therefore, recombinant retroviruses can shuttle back and forth between amphotropic and ecotropic packaging cell lines in a mixed culture (referred to as ping-ponging), thus leading to an increase in proviral DNA copy number and virus titer (Bestwick et al., 1988). Some drawbacks to the ping-pong process are that transfer of packaging functions between ecotropic and anphotropic lines can lead eventually to generation of replication-competent helper virus. Also, increasing numbers of cells express both ecotropic and amphotropic envelope proteins and are therefore resistant to further infection. Moreover, cells with large numbers of proviruses are unhealthy. Thus, there is an optimum period during the ping-pong process when virus titer is high and helper virus is absent. This time period is empirically determined and is relatively constant for a given ecotropic plus amphotropic packaging line combination.

Other viral vectors may be employed as expression constructs in the present invention. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990). Lentivirus vectors are also contemplated for use in the present invention (Gallichan et al., 1998; Miyoshi et al., 1998; Kafri et al., 1999).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

VI. Adjunct Therapies and Procedures

In accordance with the present invention, it may prove advantageous to combine the methods disclosed herein with adjunct therapies or procedures to enhance the overall therapeutic effect. Such therapies and procedures are set forth in general, below. A skilled physician will be apprised of the most appropriate fashion in which these therapies and procedures may be employed.

A. Supplemental Insulin Therapy

The present invention, though designed to eliminate the need for other therapies, may work well in combination with traditional insulin supplementation. Such therapies should be tailored specifically for the individual patient given their current clinical situation, and particularly in light of the extent to which transplanted cells can provide insulin. The following are general guidelines for typical a “monotherapy” using insulin supplementation by injection.

Insulin can be injected in the thighs, abdomen, upper arms or gluteal region. In children, the thighs or the abdomen are preferred. These offer a large area for frequent site rotation and are easily accessible for self-injection. Insulin injected in the abdomen is absorbed rapidly while from the thigh it is absorbed more slowly. Hence, patients should not switch from one area to the other at random. The abdomen should be used for the time of the day when a short interval between injection and meal is desired (usually pre-breakfast when the child may be in a hurry to go to school) and the thigh when the patient can wait 30 minutes after injection for his meal (usually pre-dinner). Within the selected area systematic site rotation must be practiced so that not more than one or two injections a month are given at any single spot. If site rotation is not practiced, fatty lumps known as lipohypertrophy may develop at frequently injected sites. These lumps are cosmetically unacceptable and, what is more important, insulin absorption from these regions is highly erratic.

Before injecting insulin, the selected site should be cleaned with alcohol. Injecting before the spirit evaporates can prove to be quite painful. The syringe is held like a pen in one hand, pinching up the skin between the thumb and index finger of the other hand, and inserting the needle through the skin at an angle of 45-90° to the surface. The piston is pushed down to inject insulin into the subcutaneous space (the space between the skin and muscle), then one waits for a few seconds after which release the pinched up skin before withdrawing the needle. The injection site should not be massaged.

For day-to-day management of diabetes, a combination of short acting and intermediate acting insulin is used. Some children in the first year after onset of diabetes may remain well controlled on a single injection of insulin each day. However, most diabetic children will require 2, 3 or even 4 shots of insulin a day for good control. A doctor should decide which regimen is best suited.

One injection regimen: A single injection comprising a mix of short acting and intermediate acting insulin (mixed in the same syringe) in 1:3 or 1:4 proportion is taken 20 to 30 minutes before breakfast. The usual total starting dose is 0.5 to 1.0 units/kg body weight per day. This regimen has three disadvantages: (1) all meals must be consumed at fixed times; (2) since the entire quantity of insulin is given at one time, a single large peak of insulin action is seen during the late and early evening hours making one prone to hyopglycemia at this time; (3) as the action of intermediate acting insulin rarely lasts beyond 16-18 hours, the patient's body remains underinsulinized during the early morning hours, the period during which insulin requirement in the body is actually the highest.

Two-injection regimen: This regimen is fairly popular. Two shots of insulin are taken—one before breakfast (⅔ of the total dose) and the other before dinner (⅓ of the total dose). Each is a combination of short acting and intermediate acting insulin in the ratio of 1:2 or 1:3 for the morning dose, and 1:2 or 1:1 for the evening dose. With this regimen the disadvantages of the single injection regimen are partly rectified. Some flexibility is possible for the evening meal. Further, as the total days' insulin is split, single large peaks of insulin action do not occur hence risk of hypoglycemia is reduced and one remains more or less evenly insulinized throughout the day. On this regimen, if the pre-breakfast blood glucose is high, while the 3 a.m. level is low, then the evening dose may need to be split so as to provide short acting insulin before dinner and intermediate acting insulin at bedtime.

Multi-dose insulin regimens: The body normally produces insulin in a basal-bolus manner, i.e., there is a constant basal secretion unrelated to meal intake and superimposed on this there is bolus insulin release in response to each meal. Multi-dose insulin regimens were devised to mimic this physiological pattern of insulin production. Short acting insulin is taken before each major meal (breakfast, lunch and dinner) to provide “bolus insulin” and intermediate acting insulin is administered once or twice a day for “basal insulin.” Usually bolus insulin comprises 60% of the total dose and basal insulin makes up the remaining 40%. With this regimen you have a lot of flexibility. Both the timing as well as the quantity of each meal can be altered as desired by making appropriate alterations in the bolus insulin doses. To take maximum advantage of this regimen, one should learn “carbohydrate counting” and work out carbohydrate:insulin ratio—the number of grams of carbohydrate for which the body needs 1 unit of insulin.

B. Immunosuppressive Therapy

In accordance with the present invention, it may prove necessary to deliver an immunosuppressive therapy to a transplant recipient to prevent graft rejection. A general approach to transplant immunosuppression is to combine agents in small doses so as to get an added immunosuppressive effect, but without individual side effects of the different drugs. Commonly used agents include azathioprine, corticosteroids and cyclosporin are combined in a variety of protocols. Each has different side effects: corticosteroids stunt growth and cause a round face and impair the healing of wounds; azathioprine can inhibit the bone marrow and cause anaemia and a low white cell count; cyclosporin can cause increase in growth of hair and damage the kidney. However, when these three agents are used together in reduced doses, the patient can generally tolerate the immunosuppression quite well.

Unfortunately, acute rejection crises can still occur and are usually treated with a short course of high dose steroids or anti-lymphocyte globulin preparations. This powerful immunosuppression for rejection may lead to infection particularly with viruses, causing severe cold sores and may activate cytomegalic virus infection which can cause a temperature and specific bad effects on a variety of different organs. All immunosuppression predisposes a patient to infection and tumour formation.

Despite these difficulties, most patients tolerate organ grafts and, after a time, can be maintained with relatively low dosage of immunosuppression. With few exceptions however, stopping immunosuppression usually leads to acute rejection and chronic rejection which is difficult to detect and can occur insidiously after years of good function of a graft. Thus, the search for new immunosuppressives such as FK506, celcept mycophenolate mofital and rapamycin is important.

C. Pro-Angiogenic Therapy

Pro-angiogenic therapy, also known as “therapeutic angiogenesis,” uses angiogenic growth factors or gene therapy to stimulate blood vessel growth in tissues that require an improved blood supply. While angiogenesis is normally activated by hypoxia (decreased oxygen), many afflicted tissues are unable to respond adequately to reverse the disease processes and prevent tissue damage.

Currently, a variety of angiogenesis-stimulating modalities are being tested in clinical trials sponsored by biotechnology and pharmaceutical companies, medical centers, and the National Institutes of Health. The most prevalently discussed pro-angiogenic therapy is the use of VEGF. Another involves a topical gel of recombinant platelet-derived growth factor (rhPDGF-BB).

D. Monitoring Glucose Levels

Any person suffering from diabetes will be very familiar with the need to regularly measure blood glucose levels. Blood glucose level is the amount of glucose, or sugar, in the blood. It is also is referred to as “serum glucose level.” Normally, blood glucose levels stay within fairly narrow limits throughout the day (4 to 8 mmol/l), but are often higher after meals and usually lowest in the morning. Unfortuantely, when a person has diabetes, their blood glucose level sometimes moves outside these limits. Thus, much of a diabetic's challenge is to When one suffers from diabetes, it is important that glucose level be as near normal as possible. Stable blood glucose significantly reduces the risk of developing late-stage diabetic complications, which start to appear 10 to 15 years after diagnosis with Type 1 diabetes, and often less than 10 years after diagnosis with Type 2 diabetes.

Blood glucose levels can be measured very simply and quickly with a home blood glucose level testing kit, consisting of a measuring device itself and a test strip. To check blood glucose level, a small amount of blood is placed on the test strip, which is then placed into the device. After about 30 seconds, the device displays the blood glucose level. The best way to take a blood sample is by pricking the finger with a lancet. Ideal values are (a) 4 to 7 mmol/l before meals, (b) less than 10 mmol/l one-and-a-half hours after meals; and (c) around 8 mmol/l at bedtime.

People who have Type 1 diabetes should measure their blood glucose level once a day, either in the morning before breakfast or at bedtime. In addition, a 24-hour profile should be performed a couple of times a week (measuring blood glucose levels before each meal and before bed). People who have Type 2 diabetes and are being treated with insulin should also follow the schedule above. People who have Type 2 diabetes and who are being treated with tablets or a special diet should measure their blood glucose levels once or twice a week, either before meals or one-and-a-half hours after a meal. They should also perform a 24-hour profile once or twice a month.

The main advantage for measuring blood glucose levels of insulin-treated diabetics in the morning is that adjusted amounts of insulin can be taken if the blood glucose level is high or low, thereby reducing the risk of developing late-stage diabetic complications. Similarly, the blood glucose level at bedtime should be between 7 and 10 mmol/l. If blood glucose is very low or very high at bedtime, there may be a need to adjust food intake or insulin dose. Blood glucose should also be measured any time the patient does not feel well, or think blood glucose is either too high or too low. People who have Type 1 diabetes with a high level of glucose in their blood (more than 20 mmol/l), in addition to sugar traces in the urine, should check for ketone bodies in their urine, using a urine strip. If ketone bodies are present, it is a warning signal that they either have, or may develop, diabetic acidosis.

VII. In Vivo Uses

A. Pharmaceutically Acceptable Formulations

Where clinical applications are contemplated, it will be necessary to prepare pharmaceutical compositions of the cells in a form appropriate for transplant. The cells will generally be prepared as a composition that is essentially free of pyrogens, as well as other impurities that could be harmful to humans or animals.

One will generally desire to employ appropriate salts and buffers to render stable cells suitable for introduction into a patient. Aqueous compositions of the present invention comprise an effective amount of stable cells dispersed in a pharmaceutically acceptable carrier or aqueous medium, and preferably encapsulated.

The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. As used herein, this term is particularly intended to include biocompatible implantable devices and encapsulated cell populations. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

Under ordinary conditions of storage and use, the cell preparations may further contain a preservative to prevent growth of microorganisms. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

B. Cell-Based Delivery and Devices

The engineered cells of the present invention may be introduced into animals, including human subjects, with certain needs, such as patients with insulin-dependent diabetes. It should be pointed out that the studies of Madsen and coworkers have shown that implantation of poorly differentiated rat insulinoma cells into animals results in a return to a more differentiated state, marked by enhanced insulin secretion in response to metabolic fuels (Madsen et al., 1988). These studies suggest that exposure of engineered cell lines to the in vivo milieu may have some effects on their response(s) to secretagogues.

A method of administration involves the encapsulation of the engineered cells in a biocompatible coating. In this approach, the cells are entrapped in a capsular coating that protects the contents from immunological responses. One encapsulation technique involves encapsulation with alginate-polylysine-alginate. Capsules made employing this technique generally have a diameter of approximately 1 mm and should contain several hundred cells.

Cells may thus be implanted using the alginate-polylysine encapsulation technique of O'Shea and Sun (1986), with modifications, as later described by Fritschy et al. (1991; both references incorporated herein by reference). The engineered cells are suspended in 1.3% sodium alginate and encapsulated by extrusion of drops of the cell/alginate suspension through a syringe into CaCl₂. After several washing steps, the droplets are suspended in polylysine and re-washed. The alginate within the capsules is then reliquified by suspension in 1 mM EGTA and then rewashed with Krebs balanced salt buffer.

An alternative approach is to seed Amicon fibers with stable cells of the present invention. The cells become enmeshed in the fibers, which are semipermeable, and are thus protected in a manner similar to the micro encapsulates (Altman et al., 1986; incorporated herein by reference). After successful encapsulation or fiber seeding, the cells may be implanted intraperitoneally, usually by injection into the peritoneal cavity through a large gauge needle (23 gauge).

A variety of other encapsulation technologies have been developed that are applicable to the practice of the present invention (see, e.g., Lacy et al., 1991; Sullivan et al., 1991; WO 91/10470; WO 91/10425; WO 90/15637; WO 90/02580; U.S. Pat. No. 5,011,472; U.S. Pat. No. 4,892,538; and WO 89/01967; each of the foregoing being incorporated by reference).

Lacy et. al. (1991) encapsulated rat islets in hollow acrylic fibers and immobilized these in alginate hydrogel. Following intraperitoneal transplantation of the encapsulated islets into diabetic mice, normoglycemia was reportedly restored. Similar results were also obtained using subcutaneous implants that had an appropriately constructed outer surface on the fibers. It is therefore contemplated that engineered cells of the present invention may also be straightforwardly “transplanted” into a mammal by similar subcutaneous injection.

Sullivan et. al. (1991) reported the development of a biohybrid perfused “artificial pancreas,” which encapsulates islet tissue in a selectively permeable membrane. In these studies, a tubular semi-permeable membrane was coiled inside a protective housing to provide a compartment for the islet cells. Each end of the membrane was then connected to an arterial polytetrafluoroethylene (PTFE) graft that extended beyond the housing and joined the device to the vascular system as an arteriovenous shunt. The implantation of such a device containing islet allografts into pancreatectomized dogs was reported to result in the control of fasting glucose levels in 6/10 animals. Grafts of this type encapsulating engineered cells could also be used in accordance with the present invention.

The company Cytotherapeutics has developed encapsulation technologies that are now commercially available that are envisioned for use in the application of the present invention. A vascular device has also been developed by Biohybrid, of Shrewsbury, Mass., that can be used with the technology of the present invention. Other implantable containment apparati contemplated for use with in the application of the present invention are described in U.S. Pat. Nos. 5,626,561, 5,787,900 and 5,843,069, each of which are incorporated herein by reference.

Implantation employing such encapsulation techniques provides various advantages. For example, transplantation of islets into animal models of diabetes by this method has been shown to significantly increase the period of normal glycemic control, by prolonging xenograft survival compared to unencapsulated islets (O'Shea and Sun, 1986; Fritschy et al., 1991). Also, encapsulation will prevent uncontrolled proliferation of clonal cells. Capsules containing cells are implanted (approximately 1,000-10,000/animal) intraperitoneally and blood samples taken daily for monitoring of blood glucose and insulin.

An alternate approach to encapsulation is to simply inject glucose-sensing cells into the scapular region or peritoneal cavity (Sato et al., 1962). Implantation by this approach may circumvent problems with viability or function, at least for the short term, that may be encountered with the encapsulation strategy.

C. Treatment

An effective amount of the stable cells is determined based on the intended goal. The term “unit dose” refers to a physically discrete unit suitable for use in a subject, each unit containing a predetermined quantity of the cell composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

### VIII. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Animals. Flk-1 (KDR, VEGFR2) heterozygote mice with lacZ-tagged endothelial cells (Flk-1^{wt/lacZ}) (Shalaby et al., 1995), C57BL/6 mice, and NOD-SCID mice were obtained from the Jackson Laboratory (Bar Harbor, Me.). To identify the lacZ insert in the Flk-1 gene, the offspring of Flk-1^{wt/lacZ} mice (stock number 002938; background strain C57BL/6) were genotyped by PCR using the following primers to detect the LacZ insertion (5′ primer: ATC CTC TGC ATG GTC AGG TC; 3′ primer: CGT GGC CGT ATT CAT TTC) and the wild-type locus (5′ primer: CAA ATG TTG CTT GTC TGG TG; 3′ primer: GTC AGT CGA GTG CAC ATG TT). Transplants of mouse islets from Flk-1^{wt/lacZ} donors and human islets were performed into immunodeficient NOD-SCID mouse model from Jackson Laboratory (for additional information, see jaxmicejax.org).

Mouse islet isolation. Islets were isolated from Flk-1^{wt/wt} mice by dissection of splenic portion of the pancreas, followed by collagenase P digestion (Roche Molecular Biochemicals) as described previously (Brissova et al., 2002). In order to increase a yield of islets isolated from Flk-lacZ mice, 3 mL of collagenase P in Hanks buffered saline (0.6 mg/mL) were first directly infused into the pancreas through the bile duct. Groups of two pancreata were then digested in 6.7 mL of collagenase P (0.6 mg/mL) for 4-5 min at 37° C. using a wrist-action shaker. Islets were handpicked under microscopic guidance and washed 3 times with 10 mM PBS containing 1% mouse serum. Finally, 200-240 islets were suspended in 30 μL of the same solution and transplanted into mouse recipients immediately following the isolation procedure.

Human islets. Human islets were obtained through the Juvenile Diabetes Foundation Human Islet Distribution Program and from Dr. David M. Harlan at the Transplantation and Autoimmunity Branch of the National Institute of Diabetes and Digestive and Kidney Diseases of the National Institutes of Health. After isolation, human islets were shipped in CMRL media by overnight courier to Vanderbilt University and cultured for additional 24 hrs in CMRL media, 95% CO₂/5% O₂ at 37° C. After culture, 500-2000 islets were transplanted into NOD-SCID mice (approximately 48 hrs after islet isolation).

Islet transplantation model. The mice were anesthetized by an intraperitoneal injection of sodium pentobarbital (50 mg/kg body weight, Abbott Laboratories, North Chicago, Ill.). After adequate anesthesia, the left flank was shaved, prepped, and draped in sterile fashion. With a left flank incision, the left kidney was identified, exposed, and irrigated with saline. The islet suspension (30 μL) was injected between the capsule and renal parenchyma of the left kidney using a 23-gauge butterfly needle. After withdrawal of the needle, the insertion point was cauterized and the wound was closed with subcutaneous sutures (Prolene, size 7-0 with cutting needle, Ethicon, Somerville, N.J.) and skin staples (Autoclips, 9 mm size, Clay Adams, Parsippany, N.J.).

Tissue collection. Adult pancreata, as well as kidneys bearing islet transplants (3-5 weeks after transplantation), were dissected in ice-cold 10 mM PBS and fixed in freshly prepared 4% paraformaldehyde (Electron Microscopy Sciences, Washington, Pa.)/100 mM PBS for 1.5 h on ice. Following fixation, the tissues were washed 4-6 times with 100 mM PBS over a period of 2 hrs and then equilibrated in 30% sucrose/10 mM PBS overnight at 4° C. The tissues were cryopreserved in optimum cutting temperature (OCT) compound (VWR Scientific Products, Willard, Ohio) at −80° C. and 10 or 60 μm sections were mounted on charged slides.

Detection of β-galactosidase activity. In case of the whole pancreas, the fixed tissue was permeabilized twice for 30 min at room temperature in permeabilization solution (2 mM MgCl₂, 0.01% sodium deoxycholate, 0.02% Nonidet P-40 in 10 mM PBS). β-Galactosidase activity was detected by incubating the tissue in staining solution (2 mM MgCl₂, 5 mM K ferricyanide, 5 mM K ferrocyanide, 100 mM Tris pH 7.3, and 1 mg/ml 5-bromo-4-chloro-3-indolyl-β-D-galactopyranoside (X-gal, Research Products International Corp., Mt. Prospect, Ill.)) overnight in the dark at room temperature. Tissue was then rinsed with 10 mM PBS, post-fixed in fresh ice-cold 4% paraformaldehyde/10 mM PBS for 1 h at 4° C., and rinsed three times with 10 mM PBS. Whole mount images of the pancreas were collected on an Olympus SZX9 microscope with an Olympus pm-C35 camera using Kodak Elite Chrome 160T film.

β-Galactosidase activity was also detected on 10 μm cryosections prepared as described above. The cryosections were post-fixed with 0.2% glutaraldehyde/1% paraformaldehyde (Electron Microscopy Sciences) for 15 min at room temperature, washed 3 times for 5 min with 10 mM PBS, and permeabilized with permeabilization solution for 10 min at room temperature. Sections were incubated with X-gal staining solution in a humidified chamber overnight at 37° C., rinsed three times with 10 mM PBS, and mounted with AquaPoly/Mount (Polysciences, Inc., Warrington, Pa.). Immunocytochemistry. Ten μm cryosections were permeabilized in 0.2% Triton X-100 for 10 min at room temperature, blocked with 5% normal donkey serum (Jackson ImmunOResearch Laboratories, Inc., West Grove, Pa.) for 1.5 h, and then incubated with primary antibodies overnight at 4° C. Secondary antibodies were applied to the tissue sections for 1 h at room temperature. Both primary and secondary antibodies were diluted in 10 mM PBS containing 1% BSA and 0.1% Triton X-100. Digital images of the 10 μm cryosections mounted with AquaPoly/Mount were acquired with a MagnaFire™ digital camera (Optronics) connected to an Olympus BX-41 fluorescence microscope.

Freshly isolated islets were attached to MatTek dishes (Cat. # P35G-0-14-C, MatTek Corporation, Ashland, Mass.) pre-coated with CELL-TAK adhesive (Becton Dickinson Labware, Bedford, Mass.) and fixed in 4% paraformaldehyde/10 mM PBS for 25 min at room temperature. The fixation was followed by three 30 min washes in 10 mM PBS and 3-h permeabilization with 0.3% Triton-X 100/10 mM PBS. The islets were blocked with 5% normal donkey serum/0.15% Triton-X 100/10 mM PBS overnight at 4° C. and then equilibrated in antibody dilution buffer twice for 20 min at room temperature. The primary and secondary antibodies were diluted in 1% BSA/0.2% Triton X-100/10 mM PBS and the incubations were carried out for 24 hrs at 4° C. Sixty μm cryosections of the islet transplants under the kidney capsule were permeabilized, blocked and stained under the same conditions as isolated islets. The islets and tissue sections were mounted with AquaPoly/Mount (Polysciences). Samples were subjected to optical sectioning using a Zeiss LSM410 or LSM510 META confocal laser scanning microscopes. Digital images were analyzed and 3-D reconstructed using MetaMorph 5.0 software (Universal Imaging Corporation, Downington, Pa.).

Antibodies. Rat anti-mouse CD31 (PECAM-1) (1:200) and mouse anti-human CD31 (1:50) monoclonal antibodies were from BD Biosciences Pharmigen. Guinea pig anti-human insulin IgG (1:1000) was from Linco Research, sheep anti-somatostatin IgG (1:1000) was from American Research Products, Inc., rabbit anti-Flk-1 IgG (1:500) was a gift from Rolf Brekken at The Hope Heart Institute, and rabbit anti-β-galactosidase IgG (1:5000) was from ICN Pharmaceuticals, Inc. The antigens were visualized using appropriate secondary antibodies conjugated with Cy2, Cy3, and Cy5 fluorophores from Jackson ImmunoResearch Laboratories, Inc. Secondary antibodies were used at concentrations recommended by manufacturer.

Lectin infusion and graft assessment. At 5 weeks after transplantation, mice transplanted with Flk-1^{wt/lacZ} islets were anesthetized with sodium pentobarbital (80 mg/kg body weight, Abbott Laboratories). Fluorescein (FITC)-conjugated tomato lectin (Lycopersicon esculentum, 1 mg/mL, Vector Laboratories) was injected into the jugular vein (0.1 mL/mouse) and allowed to circulate for 3 minutes, after which animals were sacrificed. Kidneys bearing islet transplants were dissected and preserved for cryosectioning as described above. The permeabilization step was omitted since detergent destabilizes lectin binding. Ten μm cryosections were blocked with 5% NDS/10 mM PBS/1 mM Ca²⁺ for 15 min. Incubations with primary and secondary antibodies were carried out at room temperature for 1 hr and 30 min, respectively. Both primary and secondary antibodies were diluted in 10 mM PBS containing 1% NDS and 1 mM Ca²⁺. Washes were performed using the same buffer. Sections were mounted with Vectashield mounting medium (Vector Laboratories) and subjected to optical sectioning using a LSM510 META confocal laser scanning microscope.

### Example 2

### Results

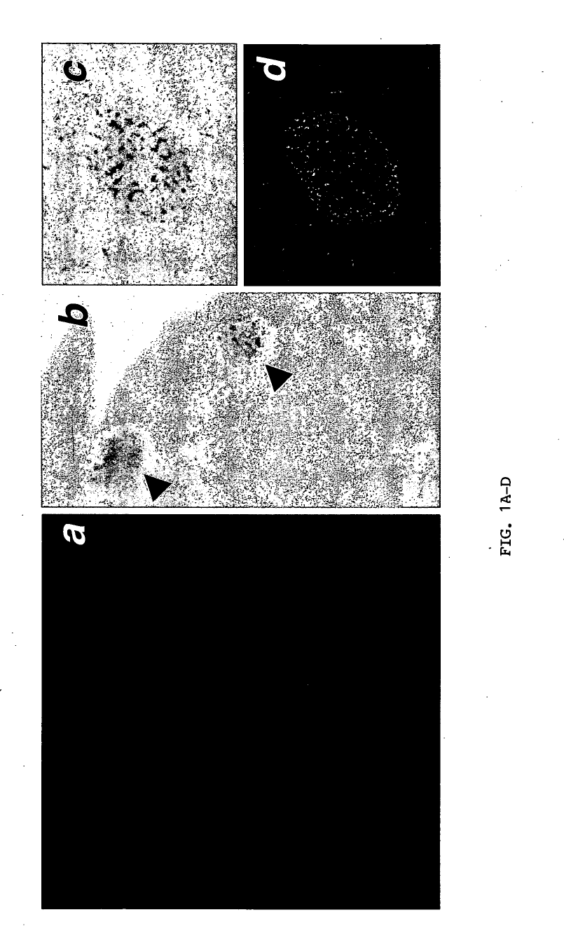

Expression of endothelial cell markers in isolated islets and pancreas of Flk-1^{wt/lacZ} mice. Even though islet isolation severs arterial and venous connections, isolated islets retain their capillary network (FIG. 1A). Therefore, the inventors asked whether these intra-islet endothelial cells contribute to the revascularization of transplanted islets. To follow the fate of the intra-islet endothelial cells, a model in which endothelial cells are tagged with lacZ (knock-in of lacZ to the Flk-1 locus termed Flk-1^{wt/lacZ}) was employed. LacZ encodes the β-galactosidase enzyme. FIG. 1B shows prominent X-gal staining (reflecting β-galactosidase activity) of islets in the whole-mount Flk-1^{wt/lacZ} pancreas. Pancreatic sections in FIGS. 1C and 1D demonstrate that lacZ expression recapitulates expression of Flk-1. Similar to Flk-1 expression, there was a greater density of lacZ⁺ capillary structures in the islets compared to exocrine tissue reflecting the higher vascularity of islets (FIGS. 1C and 1D).

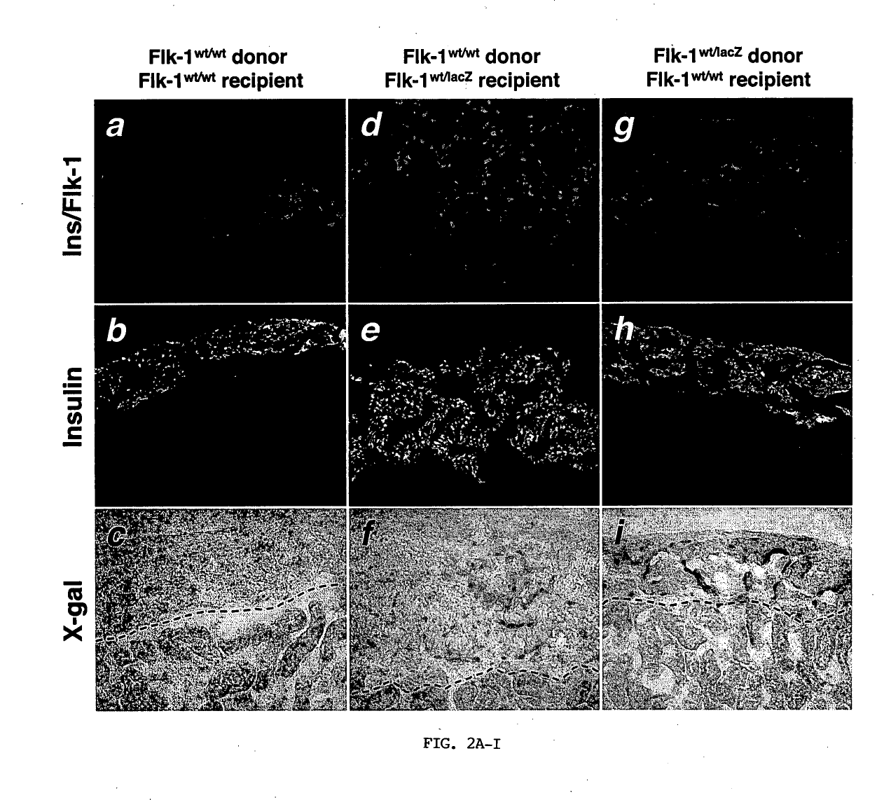

Role of intra-islet endothelial cells in revascularization process of transplanted mouse islets. Immediately after islet isolation, wild-type islets (Flk-1^{wt/wt}) were transplanted into wild type animals (Flk-1^{wt/wt}, n=3, FIGS. 2A-C) and mice that contain the endothelial lacZ tag (Flk-1 lacz, n=4, FIGS. 2D-F). At the same time, murine islets containing tagged endothelial cells (Flk-1^{wt/lacZ}) were transplanted into mice that do not carry the endothelial cell marker (n=10, FIG. 2G-I). The inventors used NOD-SCID mice as the recipient of Flk-1^{wt/lacZ} islets to avoid a possible immune reaction to endothelial cells expressing lacZ.

Kidneys bearing transplanted islets were retrieved 3-5 weeks after transplantation, a time in which the revascularization is completed as shown by previous studies (Griffith et al., 1977; Jansson & Carlsson, 2002; Lukinius et al., 1995; Menger et al., 1989; 2001; Rooth et al., 1989; Vajkoczy et al., 1995; Vajkoczy et al., 1995). In all three types of transplants, the islets were vascularized as detected by Flk-1 expression in vascular structures within and surrounding the islet graft (FIGS. 2A, 2D and 2G). As shown by detection of β-galactosidase activity (FIGS. 2F and 21), both donor and recipient endothelial cells were found within the islet graft area positive for insulin (FIGS. 2E and 2H). Occasionally, there were a few lacZ⁺ intra-islet endothelial cells migrating further away from the transplant into kidney cortex (FIG. 2I). These data indicate that intra-islet endothelial cells survive and possibly contribute to the revascularization process.

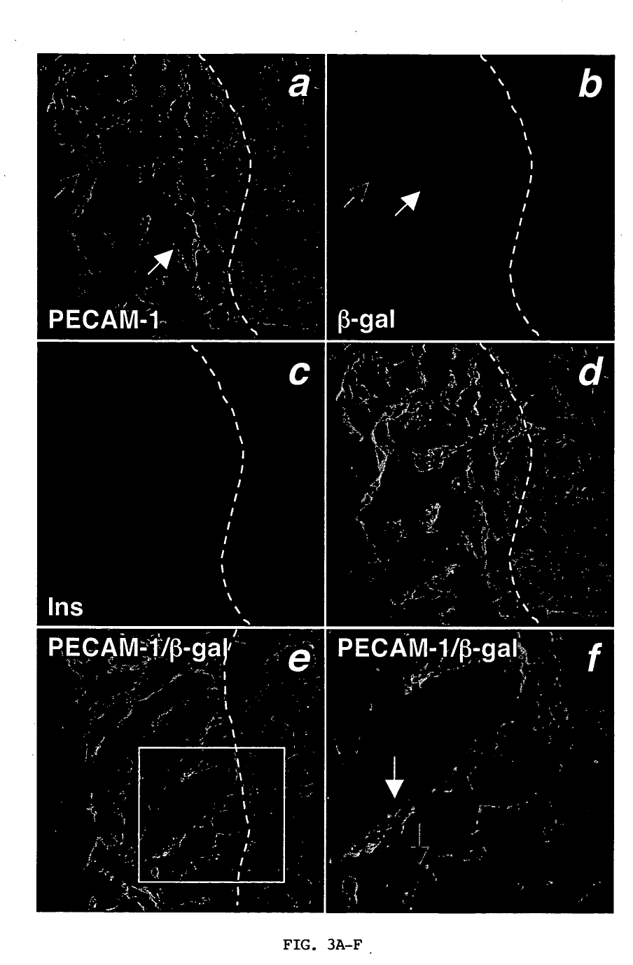

To determine whether intra-islet endothelial cells participate in revascularization, and in order to access the structure and composition of blood vessels in the revascularized grafts, 60 μm sections of the islet grafts were labeled for the mouse endothelial marker PECAM-1 that is ubiquitously expressed on the surface of all (both donor and recipient) endothelial cells. The sections were co-labeled for lacZ-encoded α-galactosidase that is only expressed by endothelium of donor Flk-1^{wt/lacZ} islets (FIGS. 2G, 2H and 2I) and unlike PECAM-1 has a more cytoplasmic localization. In both mouse and human native islets and islet transplant grafts, mouse PECAM-1 or human CD31 and Flk-1 are co-expressed in islet microvasculature (data not shown). Mounted sections were then subjected to optical sectioning using a laser scanning confocal microscope. The three dimensional reconstruction of optical sections through the islet grafts (FIG. 3) indicated the existence of two types of blood vessels in the revascularized islet graft: 1) capillaries formed predominantly of either donor or recipient endothelial cells directly connected to each other; and 2) chimeric blood vessels formed from a mixture of donor and recipient endothelial cells. By examining optical sections of the islet grafts in three dimensions, both donor and recipient endothelial cells were found to be components of tubular structures consistent with vessels that traversed throughout the islet graft (FIGS. 3E and 3F). To estimate the contribution of donor and recipient endothelial cells to the graft revascularization, we used MetaMorph software and calculated the volume of PECAM-1⁺ and β-galactosidase⁺ endothelial cells in the insulin⁺ graft area. This calculation was applied to 4 different grafts and 3-6 fields were examined per each graft. The data suggested that as much as 40±3% (n=18, range from 18 to 68%) of endothelial cells in the revascularized graft originated from the donor islets.

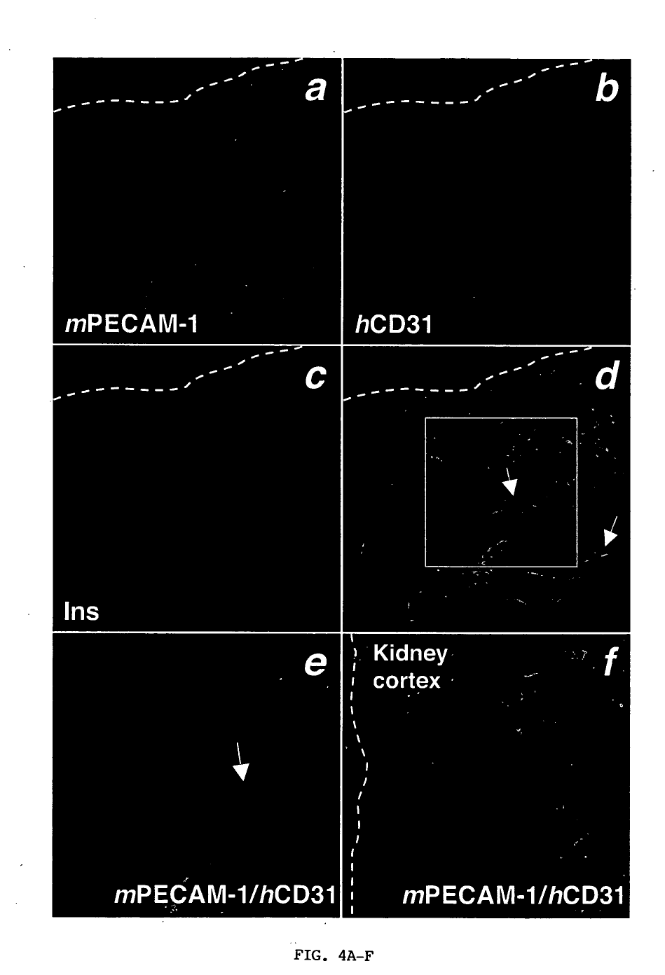

Role of intra-islet endothelial cells in revascularization process of transplanted human islets. Human islets, transplanted under the kidney capsule of NOD-SCID mice (n=4), were analyzed for the presence of the donor and recipient endothelial cells using species-specific antibodies to the endothelium-specific marker CD31. Sixty-μm sections of the human islet grafts were stained for mouse CD31 (PECAM-1) and human CD31. Mounted sections were then subjected to optical sectioning using a laser scanning confocal microscope. The three dimensional reconstruction of optical sections through the islet grafts (FIG. 4) indicated that human intra-islet endothelial cells, similar to the murine islets, survive after transplantation. In contrast to mouse intra-islet endothelial cells, the human intra-islet endothelial cells did not form chimeric blood vessels. Moreover, examination of human islet grafts in three dimensions revealed fewer possible connections of human endothelial cells with the host vasculature (FIGS. 4D and 4E) than was seen in mouse islet grafts. The human intra-islet endothelial cells also appeared to more readily migrate into the kidney cortex (FIG. 4F) than was observed in transplants of murine Flk-1^{wt/lacZ} islets (FIG. 2I).

D intra-islet endothelial cells become a part of functional vasculature in revascularized graft? Even though intra-islet endothelial cells survived transplantation and were a part of vascular-like structures within the revascularized islet grafts, it was not known whether they integrated into a functional blood vessel network of the revascularized graft. To address this question, mice that received transplants of Flk-1^{wt/lacZ} islets were infused with FITC-conjugated tomato lectin (n=4), which binds with high affinity to the surface of mouse endothelial cells (Inoue et al., 2002). Lectin infusion, a well-established technique, has been widely used in the area of angiogenesis (McDonald & Choyke, 2003). For example, many investigators feel that it is the technique of choice for identifying functional blood vessels, since the only way the lectin can reach the endothelial cells is via the circulation (McDonald & Choyke, 2003). Furthermore, it has been shown previously that Lycopersicon esculentum lectin, utilized in the inventors' studies, binds uniformly to luminal surface of murine endothelial cells in arteries, veins and capillaries and is co-localized with CD31 in functional blood vessels (McDonald & Choyke, 2003). The inventors confirmed the uniformity of Lycopersicon esculentum lectin binding in both pancreas and kidney. Optical sections of the lectin-perfused mouse tissue specimens acquired with laser scanning confocal microscope show that the lectin signal is localized in the luminal lining of blood vessels.

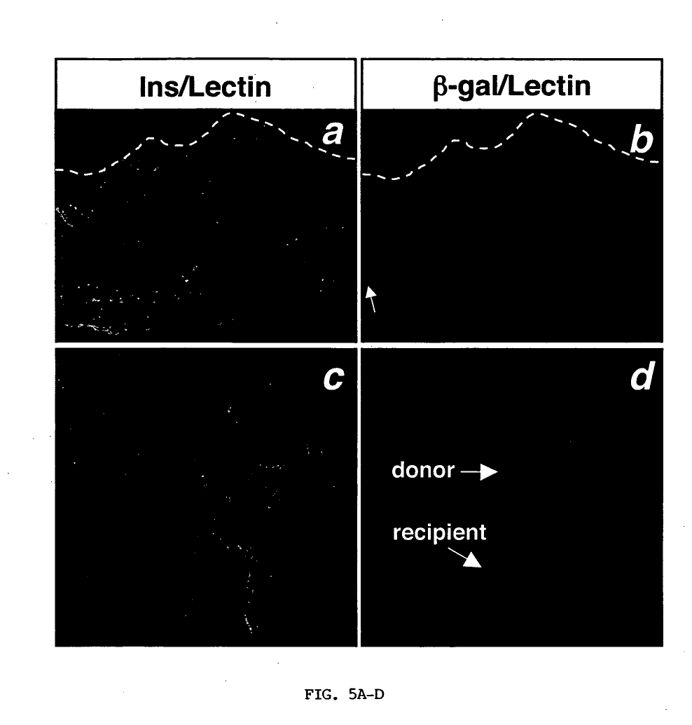

The lectin-perfused islet grafts were additionally analyzed for expression of insulin and β-galactosidase by immunocytochemistry and laser scanning confocal microscopy (FIG. 5). The series of optical sections was acquired at a 0.5 μm interval in the axial (z) dimension and an appropriate pinhole setting to alleviate any concerns about overlapping signals from the layers of the specimen above and below the given focal plane. Individual optical sections of the islet grafts demonstrate that in the same focal plane, β-galactosidase, which marks intra-islet endothelial cells, is co-localized with lectin thus proving unequivocally the functionality of these β-galactosidase-positive blood vessels. The presence of cells double-positive for lectin and β-galactosidase proves indeed that blood flows through donor-derived blood vessels in revascularized mouse islet grafts (FIGS. 5B and 5D). Immunocytochemistry of the lectin-perfused specimens required several modifications to avoid lectin leaching during the staining procedure (see Methods) and underestimated the number of α-galactosidase⁺ endothelial cells (especially in cells with lower β-galactosidase expression). This was based on comparison of β-galactosidase⁺ cells in consecutive sections stained either with β-galactosidase antibody (modified immunocytochemistry procedure) or using enzymatic reaction of β-galactosidase with X-gal substrate. As there were fewer β-galactosidase⁺ cells detected by immunocytochemistry compared to enzymatic reaction, the inventors did not believe it appropriate to estimate the ratio of β-galactosidase⁺ cells to cells double-positive for β-galactosidase⁺ and lectin⁺. They did observe a few cells positive for β-galactosidase and negative for lectin and for example, one such cell appeared in FIG. 5B (open arrowhead). These β-galactosidase⁺/lectin⁻ cells could be either intra-islet endothelial cells that survived the transplantation, but did not establish a lumen or proliferating donor-derived endothelial cells.

In the human islet grafts perfused with Lycopersicon esculentum lectin, they did not find human endothelial cells double positive for human CD31 and lectin. While binding of Lycopersicon esculentum lectin to mouse endothelium is well documented, it is unknown if this lectin binds to human endothelial cells (McDonald & Choyke, 2003). The human CD31+cells remained negative for the lectin even when the sections of human islet grafts were subsequently stained with Lycopersicon esculentum lectin (a standard method to assess if a lectin binds to endothelial cells). These observations indicate that human intra-islet endothelial cells may lack the N-acetyl-D-glucosamine olygomer moieties that are recognized by Lycopersicon esculentum lectin. The formation of tubular structures by human endothelial cells is evident from the 3-D projections in FIG. 4; however, it is still possible that they were incompletely connected to recipient's vasculature.

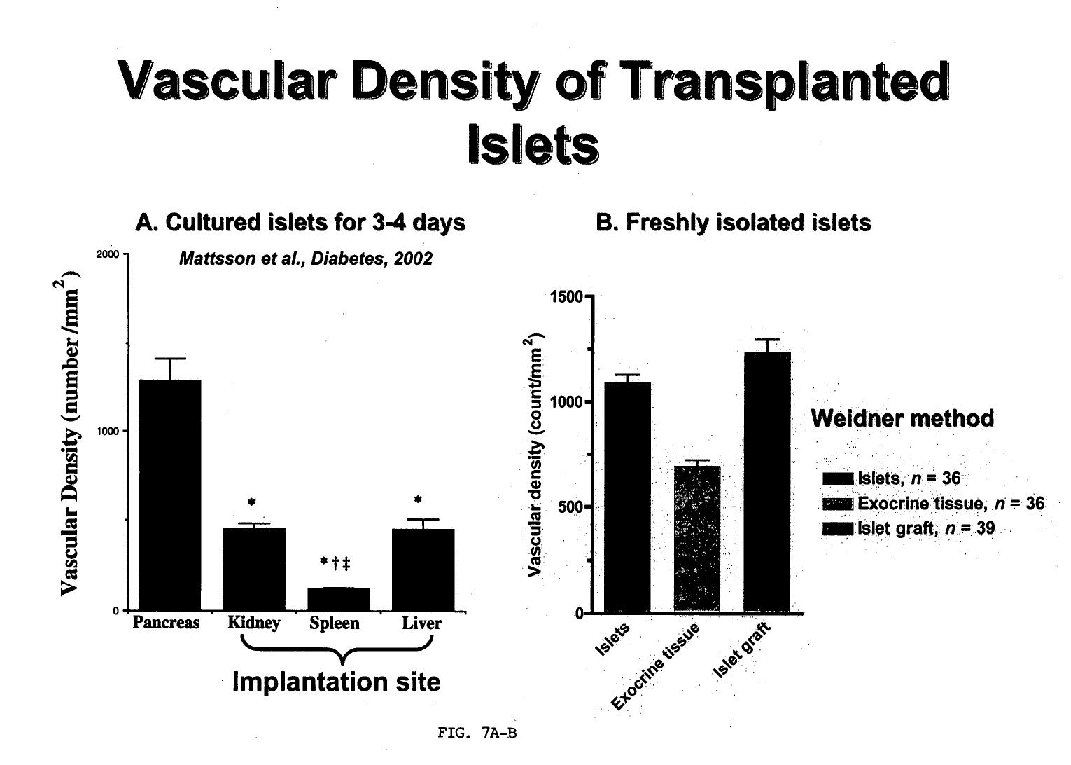

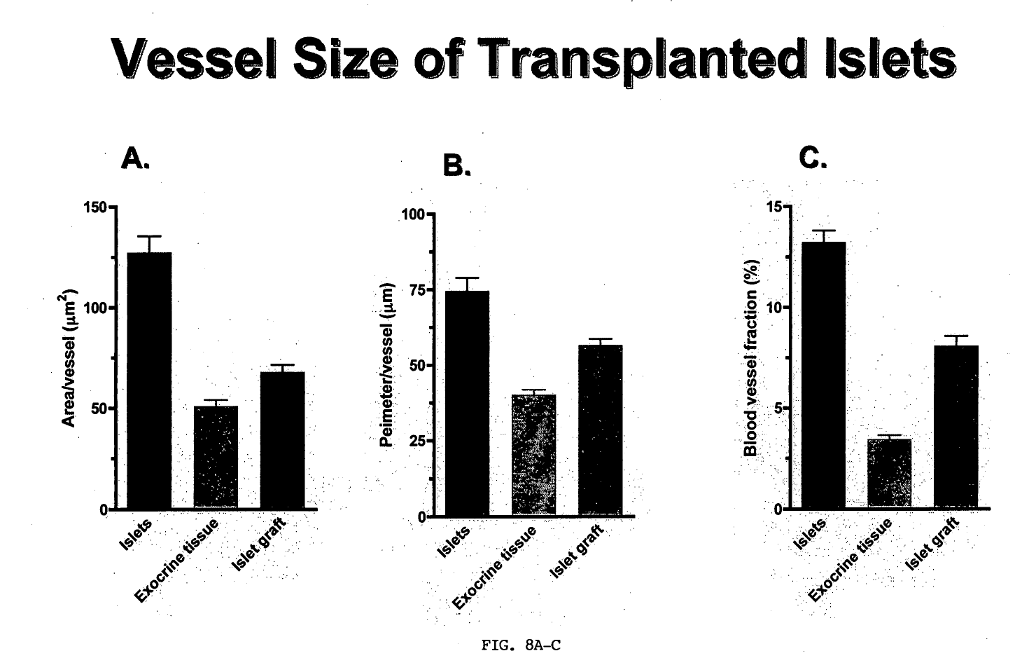

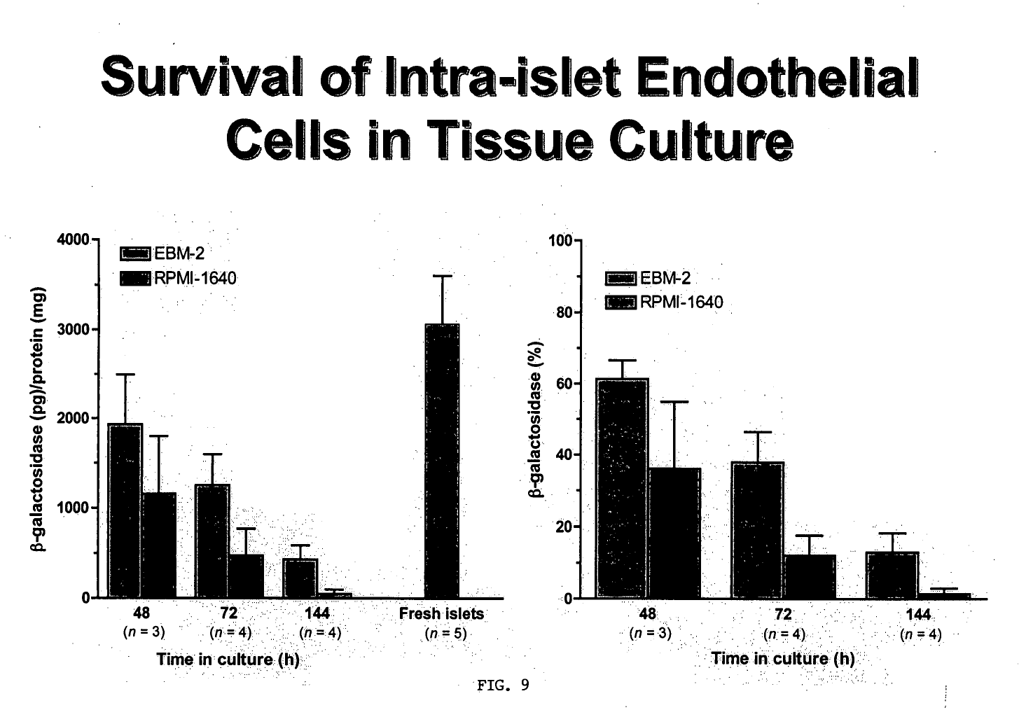

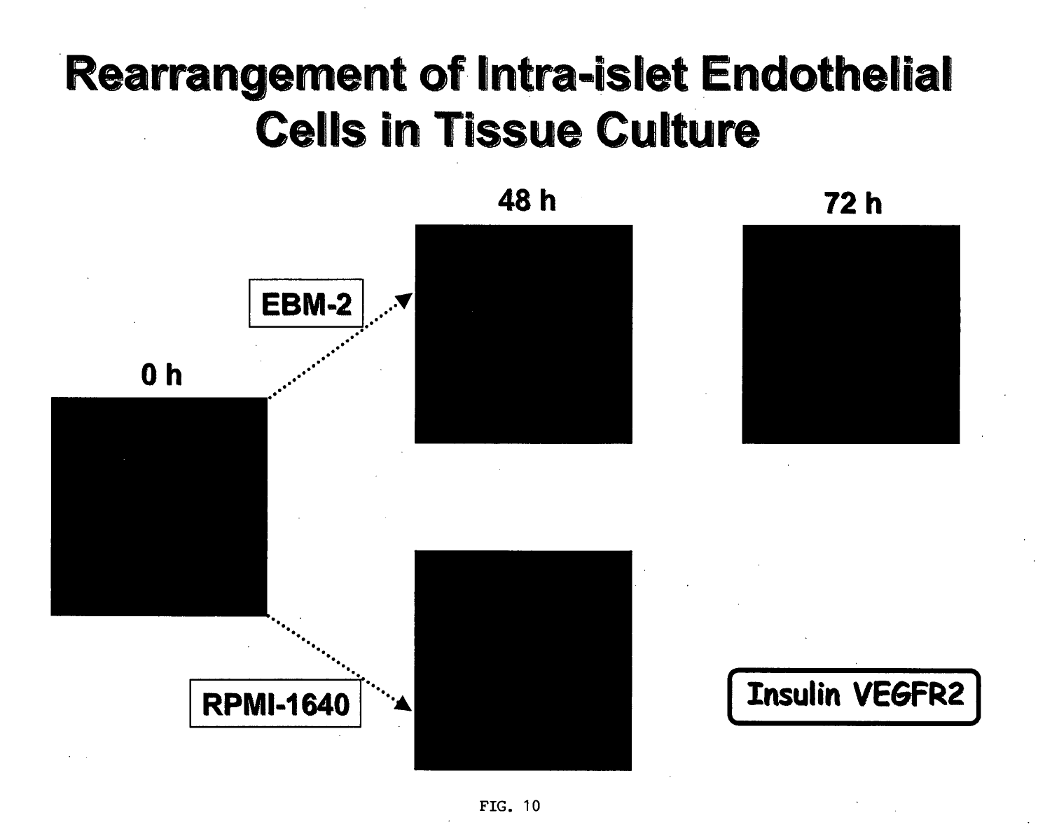

What is the role of intra-islet endothelial cells in the revascularization of transplanted islets? To examine the role of intra-islet endothelial cells in the revascularization of transplanted islets, the inventors used a murine model in which endothelial cells wre tagged with lacZ (knock-in of LacZ to the Flk-1/KDRIVEGFR2 gene). Five weeks after transplantation of freshly isolated islets with lacZ-tagged endothelial cells into NOD-SCID mice (n=4), animals were infused with the endothelium-binding Lycopersicon esculentum lectin-FITC and the vascular density of transplanted islets was examined using the Weidner method. Grafts of freshly isolated islets had a vascular density similar to that of native islets in the pancreas (1237+61 vs. 1091±37 vessels/mm²). FIG. 7B. However, vascularization parameters within islet grafts such as area/vessel (68±3 μm²) and perimeter/vessel (57±2 μm) were more similar to those in exocrine tissue (51±3 μm² and 40±2 μm) indicating that vessels within revascularized islet grafts are reduced in size and/or branching compared to the native islets in the pancreas (area/vessel—127±8 μm²; perimeter/vessel—75±4 μm). FIG. 8A-C. Since most islet transplantation programs are now culturing islets before transplantation, the inventors also asked whether culture affects the viability of intra-islet endothelial cells. Pancreatic islets with lacZ-tagged endothelial cells were cultured for 2-6 days either in medium optimized for endocrine cells (RPMI-1640) or endothelial cell-optimized medium (Endothelial Cell Basal Medium-2; EBM-2). β-galactosidase expression, as an indicator of intra-islet endothelial cell survival in culture, was measured by ELISA in islet lysates. β-galactosidase expression markedly declined during culture, but was better maintained by culture in the endothelial cell-optimized medium as compared to RPMI (2 days—61±5% vs. 36±19%; 3 days—38±8% vs. 12±5%; 6 days—13±5% vs. 1%; n=3-4; data expressed as percentage of β-galactosidase expression in freshly isolated islets). FIGS. 9-10. These results suggest that (1) revascularized islets have a different vasculature than native islets; and (2) intra-islet endothelial cell health declines during islet culture and this may reduce the contribution of intra-islet endothelial cells to the revascularization of transplanted islets.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. REFERENCES

The following references, to the extent that they provide exemplary procedural or other details supplementary to those set forth herein, are specifically incorporated herein by reference.
U.S. Pat. No. 4,195,125
U.S. Pat. No. 4,682,195
U.S. Pat. No. 4,683,202
U.S. Pat. No. 4,797,368
U.S. Pat. No. 4,892,538
U.S. Pat. No. 5,011,472
U.S. Pat. No. 5,139,941
U.S. Pat. No. 5,336,615
U.S. Pat. No. 5,427,940
U.S. Pat. No. 5,587,301
U.S. Pat. No. 5,626,561
U.S. Pat. No. 5,783,669
U.S. Pat. No. 5,787,900
U.S. Pat. No. 5,843,069
EPO 0273085
PCT/US99/00553
WO 87/05929
WO 89/01967
WO 90/02580
WO 90/06997
WO 90/15637
WO 91/09939
WO 91/10425
WO 91/10470
WO 95/29989
Ahlgren et al., Nature 385:257-260, 1997.
Altman et al., Diabetes, 35:625-633, 1986.
Baichwal et al., In: Gene transfer, Kucherlapati (Ed), Nyork, Plenum Press, 117-148, 1986.
Bauer et al., Blood 86:2379-2387, 1995.
Beattie et al., J. Clin. Endocrinol. Metab., 73:93-98, 1991.
Becker et al., J. Biol. Chem., 269(33):21234-8, 1994a.
Becker et al., Methods Cell Biol., 43 Pt A:161-89, 1994b.
Becker et al., J. Biol. Chem., 271:390-394, 1996.
Bestwick et al., Proc. Natl. Acad. Sci. USA, 85:5404-5408, 1988.
Biancone et al., Cell Transplant, 11:309-311, 2002.
Brissova et al., Journal of Biological Chemistry., 277:11225-11232, 2002.
Carlsson et al., Cell Transplant., 11:813-820, 2002.
Carlsson et al., J Clin Endocrinol Metab., 87:5418-5423, 2002.
Carlsson et al., Upsala Journal of Medical Sciences, 105:107-123, 2000.
Cavallo et al., Immunology, 75:664-668, 1992.
Chang et al., Hepatology, 14:134A, 1991.
Chen et al., Mol. Cell Biol., 7:2745-2752, 1987.
Clark et al., Endocrinology, 126(4):1895-903, 1990.
Clark et al., Endocrinology, 127(6):2779-88, 1990.
Clark et al., Human Gene Therapy, 6:1329-1341, 1995.
Coffin, In: Virology, Fields et al. (Eds.), NY, Raven Press, 1437-1500, 1990.
Comi et al., In: Principles and Practice of Endocrinology and Metabolism, Becker (Ed.), Lippincott Co., Philadelphia, Chap. 152, 1346, 1995.
Cotton et al., Proc. Natl. Acad. Sci. USA, 89:6094-6098, 1992.
Couch et al., Am. Rev. Resp. Dis., 88:394-403, 1963.
Coupar et al., Gene, 68:1-10, 1988.
Culliton, Science 246:746-751, 1989.
Curiel, In: Viruses in Human Gene Therapy, Vos (Ed.), Carolina Academic Press, Durham, N.C., 179-212, 1994.
Davalli et al., Diabetes, 44:104-111, 1995.
Davalli et al., Diabetes, 45:1161-1167, 1996.
Davalli et al., Transplantation Proceedings, 27:3238-3239, 1995.
Dichek et al., Circulation 80:1347-1353, 1989.
Efrat et al., Proc. Natl. Acad. Sci. USA, 85(23):9037-41, 1988.
Efrat et al., Diabetes, 42(6):901-7, 1993.
Fechheimer et al., Proc. Natl. Acad. Sci. USA, 84:8463-8467, 1987.
Ferkol et al., FASEB J, 7:1081-1091, 1993.
Flotte et al., Am. J. Respir. Cell Mol. Biol., 7:349-356, 1992.
Flotte et al., Proc. Natl. Acad. Sci. USA, 90:10613-10617, 1993.
Flotte et al., Gene Therapy, 2:29-37, 1995.
Flowers et al., Proc. Natl. Acad. Sci. USA, 87:2349-2353, 1990.
Fraley et al., Proc. Natl. Acad. Sci. USA, 76:3348-3352, 1979.
Fritschy et al., Diabetes, 40:37, 1991.
Gainer et al., Transplantation, 61(11):1567-1571, 1996.
Gallichan et al., Hum. Gene Ther., 9:2717-2726, 1998.
German et al., Genes Dev. 6:2165-2176, 1992b.
German et al., Genomics 24:403-404, 1994.
German et al., J. Biol. Chem., 265(36):22063-6, 1990.
German et al., Mol. Cell Biol., 12(4):1777-1788, 1992a.
Ghosh-Choudhury et al., EMBO J., 6:1733-1739, 1987.
Ghosh et al., In: Liver diseases, targeted diagnosis and therapy using specific receptors and ligands, Wu and Wu (Ed.), NY, Marcel Dekker, 87-104, 1991.
Gomez-Foix et al., J. Biol. Chem., 267:25129-25134, 1992.
Gopal, Mol. Cell Biol., 5:1188-1190, 1985.
Graham and van der Eb, Virology, 52:456-467, 1973.
Graham et al., J. Gen. Virol., 36:59-72, 1977.
Graham et al., Prevec, In: Methods in Molecular Biology: Gene Transfer and Expression Protocol, Murray (Ed.), Clifton, N.J., Humana Press, 7:109-128, 1991.
Graham et al., Biotechnology, 20:363-390, 1992.
Griffith et al., Diabetes, 26:201-214, 1977.
Grunhaus et al., Seminar in Virology, 3:237-252, 1992.
Gueli et al., J. Exp. Clin. Cancer Res., 6:281, 1987.
Harland and Weintraub, J. Cell Biol., 101:1094-1099, 1985.
Hayek et al., Diabetes, 44(12):1458-60, 1995.
Hermonat and Muzyczka, Proc. Natl. Acad. Sci. USA, 81:6466-6470, 1984.
Hersdorffer et al., DNA Cell Biol., 9:713-723, 1990.
Herz and Gerard, Proc. Natl. Acad. Sci. USA, 90:2812-2816, 1993.
Hirshberg et al., J Clin Endocrinol Metab., 87:5424-5429, 2002
Horwich et al. J. Virol., 64:642-650, 1990.
Inoue et al., Cancer Cell., 1:193-202., 2002.
Inoue et al., Genomics 40:367-370, 1997.
Jang et al., J. Virol., 62(8):2636-2643, 1988.
Jansson et al., Diabetologia, 45:749-763, 2002.
Johnson et al., Mol. Cell. Biol. 17:3488-3496, 1997.
Jones and Shenk, Cell, 13:181-188, 1978.
Jonsson et al., Nature, 371:606-609, 1994.
Kafri et al., J. Virol. 73:570-584, 1999.
Kaneda et al., Science, 243:375-378, 1989.
Kaplitt et al., Nature Genetics, 8:148-154, 1994.
Karlsson et al., EMBO J, 5:2377-2385, 1986.
Kato et al., J. Biol. Chem., 266:3361-3364, 1991.
Kelleher and Vos, Biotechniques, 17(6):1110-1117, 1994.
Kiem et al., Blood 83:1467-1473, 1994.
Klein et al., Nature, 327:70-73, 1987.
Knaack et al., Diabetes, 43(12):1413-7, 1994.
Kotin et al., Proc. Natl. Acad. Sci. USA, 87:2211-2215, 1990.
Kruse et al., Genes and Dev., 7:774-786, 1993.
Lacy et al., Science, 254:1782-1784, 1991.
Lacy, Sci. Am., 273(1):50-1, 54-8, 1995.
LaFace et al., Virology, 162:483-486, 1988.
Lammert et al., Science., 294:564-567, 2001.
Laughlin et al., J. Virol., 60:515-524, 1986.
Le Gal La Salle et al., Science, 259:988-990, 1993.
Lebkowski et al., Mol. Cell. Biol., 8:3988-3996, 1988.
Leonard et al., Mol Endocrinol, 7:1275-1283, 1993.
Levrero et al., Gene, 101: 195-202, 1991.
Liang et al., Gene Therapy, 3:350-356, 1996.
Lukinius et al., American Journal of Pathology, 146:429-435, 1995.
Luo et al., Blood, 82(Supp.): 1,303A, 1994.
Macejak and Sarnow, Nature, 353:90-94, 1991.
Madsen et al., Proc. Natl. Acad. Sci. USA, 85:6652-6656, 1988.
Mann et al., Cell, 33:153-159, 1983.
Markowitz et al., J. Virol., 62:1120-1124, 1988.
Menger et al., Diabetes, 38:199-201, 1989.
Menger et al., World J Surg., 25:509-515, 2001.
McCarty et al., J. Virol., 65:2936-2945, 1991.
McDonald et al., Nat Med., 9:713-725., 2003.
McLaughlin et al., J. Virol., 62:1963-1973, 1988.
Melloul et al., Proc. Natl. Acad. Sci. USA, 90(9):3865-9, 1993.
Miller and Rosman, BioTechniques, 7:980-988, 1989.
Miller et al., Meth. Enzymol., 217:581-599, 1993.
Miller et al., EMBO J, 13:1145-1156, 1994.
Miyazaki et al., Endocrinology, 127(1):126-32, 1990.
Miyoshi et al., J. Virol., 72:8150-8157, 1998.
Muzyczka, Curr. Top. Microbiol. Immunol., 158:97-129, 1992.
Nabel et al., Science, 244:1342-1343, 1989.
Nabel et al., Science, 249:1285-1288, 1990.
Nicolas and Rubinstein, In: Vectors: A survey of molecular cloning vectors and their uses, Rodriguez and Denhardt (Eds.), Stoneham, Butterworth, 494-513, 1988.
Nicolau and Sene, Biochim. Biophys. Acta, 721:185-190, 1982.
Nicolau et al., Methods Enzymol., 149:157-176, 1987.
Nistri et al., Biol. Proc. Online, 4:32-37, 2002.
Nylen and Becker, In: Principles and Practice of Endocrinology and Metabolism, Becker (Ed.), Lippincott Co., Philadelphia, 171:1472-1479, 1995.
O'Shea and Sun, Diabetes, 35:943-946, 1986.
Ohi et al., Gene, 89L:279-282, 1990.
Ohlsson et al., EMBO J, 12:4251-4259, 1993.
Osborne et al., Hum. Gene Ther., 1:31-41, 1990.
Otonkoski et al., J. Clin. Invest., 92:1459-1466, 1993.
Paskind et al., Virology, 67:242-248, 1975.
Pearse and Takor, Fed. Proc., 38:2288-2294, 1979.
Peers et al., Mol. Endocrin., 8:1798-1806, 1994.
Pelletier and Sonenberg, Nature, 334:320-325, 1988.
Perales et al., Proc. Natl. Acad. Sci. USA, 91:4086-4090, 1994.
Potter et al., Proc. Natl. Acad. Sci. USA, 81:7161-7165, 1984.
Racher et al., Biotechnology Techniques, 9:169-174, 1995.
Ragot et al., Nature, 361:647-650, 1993.
Renan, Radiother. Oncol., 19:197-218, 1990.
Rich et al., Hum. Gene Ther., 4:461-476, 1993.
Ricordi et al., Diabetes, 37:413-420, 1988.
Ridgeway, In: A survey of molecular cloning vectors and their uses, Rodriguez et al. (Ed.), Stoneham, Butterworth, 467-492, 1988.
Rippe et al., Mol. Cell Biol., 10:689-695, 1990.
Robertson et al., New England Journal of Medicine, 343:289-290, 2000.
Rooth et al., Diabetes, 38:202-205, 1989.
Rosenfeld et al., Science, 252:431-434, 1991.
Rosenfeld et al., Cell, 68:143-155, 1992.
Roux et al., Proc. Natl. Acad. Sci. USA, 86:9079-9083, 1989.
Ryan et al., Diabetes, 50:710-719, 2001.
Sambrook et al., “Molecular Cloning: A Laboratory Manual,” Nolan, (Ed.), Cold Spring Harbor Laboratory Press, Cold Spring Harbor, N.Y., 1989.
Samulski et al., J. Virol., 63:3822-3828, 1989.
Samulski et al., EMBOJ., 10:3941-3950, 1991.
Sander and German, J. Mol. Med. 75:327-340, 1997.
Sander et al., Genes Dev. 11: 1662-1673, 1997.
Sato et al., Proc. Natl. Acad. Sci. USA, 48:1184-1190, 1962.
Shalaby et al., Nature, 376:62-66, 1995.
Shapiro et al., New England Journal of Medicine, 343:230-238, 2000.
Shelling and Smith, Gene Therapy, 1: 165-169, 1994.
Soldevila et al., J Autoimmunity, 4:381-396, 1991.
Stockschlaeder et al., Hum. Gene Ther. 2:33-39, 1991.
Stratford-Perricaudet et al., Hum. Gene Ther., 1:241-256, 1990.
Stratford-Perricaudet and Perricaudet, In: Human Gene Transfer, Cohen-Haguenauer and Boiron (Eds.), John Libbey Eurotext, France, 51-61, 1991.
Sullivan et al., Science, 252:718-721, 1991.
Sussel et al., Development, 125:2213-2221, 1998.
Temin, In: Gene Transfer, Kucherlapati (Ed.), NY, Plenum Press, 149-188, 1986.
Top et al., J. Infect. Dis., 124:155-160, 1971.
Tratschin et al., Mol. Cell. Biol., 4:2072-2081, 1984.
Tratschin et al., Mol. Cell. Biol., 5:32581-3260, 1985.
Tur-Kaspa et al., Mol. Cell Biol., 6:716-718, 1986.
Vajkoczy et al., Am. J. Pathol., 146:1397-1405, 1995.
Vajkoczy et al., Transplantation, 60:123-127, 1995.
Wagner et al., Science, 260:1510-1513, 1990.
Walsh et al., J. Clin. Invest., 94:1440-1448, 1994.
Wei et al., Gene Therapy, 1:261-268, 1994.
Wilson et al., Science 244:1344-1346, 1989.
Wong et al., Gene, 10:87-94, 1980.
Wu and Wu, J. Biol. Chem., 262:4429-4432, 1987.
Wu and Wu, Biochemistry, 27:887-892, 1988.
Wu and Wu, Adv. Drug Delivery Rev., 12:159-167, 1993.
Yang et al., Proc. Natl. Acad. Sci. USA, 87:9568-9572, 1990.
Yang et al., J. Virol., 68:4847-4856, 1994a.
Yoder et al., Blood, 82(Supp.): 1:347A, 1994.
Zhou et al., Exp. Hematol., 21:928-933, 1993.
Zhou et al., J. Exp. Med., 179:1867-1875, 1994.
Zwiebel et al., Science 243:220-222, 1989.

1. A method for transplanting insulin-producing cells comprising:
(a) providing a cell preparation comprising insulin-producing cells;
(b) performing one or more steps that increase the quantity and/or quality of endothelial cells in said preparation; and
(c) transplanting cells of said preparation into a host mammal.

2. The method of claim 1, wherein step (b) comprises increasing the quantity of endothelial cells in said preparation.

3. The method of claim 2, wherein increasing the quantity of endothelial cells comprises one or more of (i) adding endothelial cells to said preparation, (ii) culturing endothelial cells of said preparation, or (iii) contacting endothelial cells of said preparation with one or more growth factors.

4. The method of claim 1, wherein step (b) comprises increasing the quality of endothelial cells in said composition.

5. The method of claim 4, wherein increasing the quality of endothelial cells comprises one or more of (i) genetically-modifying endothelial cells in said preparation, (ii) culturing endothelial cells of said preparation, or (iii) contacting endothelial cells of said preparation with one or more growth factors.

6. The method of claim 1, wherein step (c) comprises transplanting by injection into the host hepatic portal vein.

7. The method of claim 1, wherein said insulin-producing cells comprise islet cells, hepatocytes, neurons, myocytes or genetically engineered cells.

8. The method of claim 1, wherein said insulin-producing cells are obtained from said host, from a distinct living donor, or from a cadaver.

9. The method of claim 1, wherein said endothelial cells are intra-islet endothelial cells, stem cells or bone marrow cells.

10. The method of claim 1, wherein said endothelial cells are obtained from said host, from a distinct living donor, or from a cadaver.

11. The method of claim 1, further comprising the step of treating said host with a pro-angiogenic composition or immunosuppressive agent.

12. The method of claim 11, further comprising the step of treating said living donor with a pro-angiogenic composition.

13. The method of claim 1, further comprising the step of providing insulin to said host.

14. The method of claim 1, further comprising monitoring insulin levels in said host.

15. The method of claim 1, further comprising monitoring glucose levels in said host.

16. The method of claim 1, further comprising performing at least step (c) a second time.

17. The method of claim 1, further comprising performing at least steps (b) and (c) a second time.

18. The method of claim 1, further comprising performing steps (a)-(c) a second time.

19. The method of claim 1, further comprising genetically modifying an endothelial cell provided in said preparation or provided from another source.

20. The method of claim 1, wherein said host is a human.

21. A method for enhancing the effectiveness of transplantable insulin-producing cell preparation comprising increasing the quantity and/or quality of endothelial cells in said preparation.

22. The method of claim 21, comprising increasing the quantity of endothelial cells in said preparation.

23. The method of claim 22, wherein increasing the quantity of endothelial cells comprises one or more of (i) adding endothelial cells to said preparation, (ii) culturing endothelial cells of said preparation, or (iii) contacting endothelial cells of said preparation with one or more growth factors.

24. The method of claim 2, comprising increasing the quality of endothelial cells in said composition.

25. The method of claim 24, wherein increasing the quality of endothelial cells comprises one or more of (i) genetically-modifying endothelial cells in said preparation, (ii) culturing endothelial cells of said preparation, or (iii) contacting endothelial cells of said preparation with one or more growth factors.

26. The method of claim 21, wherein said insulin-producing cells comprise islet cells, hepatocytes, neurons, myocytes or genetically engineered cells.

27. The method of claim 21, wherein said endothelial cells are intra-islet endothelial cells, stem cells or bone marrow cells.

28. The method of claim 21, further comprising genetically modifying an endothelial cell provided in said preparation or provided from another source.

29. The method of claim 29, wherein genetically modifying said endothelial cell comprises non-viral transformation with a gene that promotes cell growth, cell differentiation or cell survival.

30. The method of claim 29, wherein genetically modifying said endothelial cell comprises viral transformation with a gene that promotes cell growth, cell differentiation or cell survival.
